(19) United States
(12) Patent Application Publication
(10) Pub. No.: US 2020/0163950 A1
(43) Pub. Date: May 28, 2020

(54) PIPERIDINE-DIONE DERIVATIVES FOR USE AS CONTRACEPTIVES

(71) Applicant: SPERMATECH AS, Oslo (NO)

(72) Inventors: Bora SIENG, Oslo (NO); Steffi LUNDVALL, Oslo (NO); Claudia Alejandra BØEN, Oslo (NO); Kathrin HNIDA, Oslo (NO); Jo KLAVENESS, Oslo (NO)

(21) Appl. No.: 16/613,855

(22) PCT Filed: May 16, 2018
(86) PCT No.: PCT/GB2018/051332

§ 371 Date: Nov. 15, 2019

(30) Foreign Application Priority Data
May 16, 2017 (GB) 1707846.0

(51) Int. Cl. A61K 31/4545; A61K 9/00; A61K 31/5377; A61P 15/16

(52) U.S. Cl. CPC A61K 31/4545; A61P 15/16; A61K 31/5377; A61K 9/0053

(57) ABSTRACT

The invention relates to a method of reducing sperm motility or of contraception in a subject, said method comprising the step of administering to said subject an effective amount of a compound of formula (I), a stereoisomer, tautomer, pharmaceutically acceptable salt or prodrug thereof: (I) wherein A₁ to A₆ and R₁ to R₄ are as defined herein.

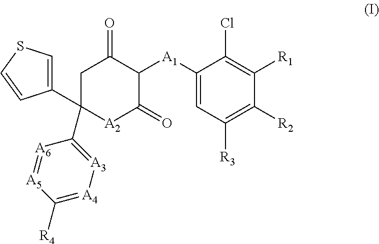

### FIELD OF INVENTION

The present invention relates to the use of piperidine-dione derivatives, and to pharmaceutical formulations containing such compounds, as sperm motility-reducing agents, e.g. for contraceptive uses.

### BACKGROUND OF INVENTION

More than 33 million of the 208 million pregnancies that occur annually worldwide are unintended, with about 20% of all unintended pregnancies ending in induced abortions. The World Health Organization (WHO) estimates that over 120 million couples do not use contraceptives due to various reasons, including side-effects of existing contraceptives for women and latex allergies to men, although many want to limit their childbearing. Many unintended pregnancies happen due to the lack of affordable, easy to use contraceptives which are free from side-effects. Thus, there is a need for alternative contraceptive methods.

It has been shown that couples with access to family-planning services often choose to have smaller families. This makes parents able to offer more time and resources to each child and thereby improve their health and quality of life. Furthermore, in the developing world, complications from pregnancy and childbirth are the leading causes of death among women of reproductive age. In 2000, the WHO estimated that 529,000 women worldwide die from such complications, 99% of them in developing countries.

Studies from the last decade show that some men want to share the responsibility for contraceptives and family planning. Additionally, there are large numbers of women who are not satisfied with their current choices of contraceptives, particularly those who have suffered from various side-effects since the pill was introduced in the 1960s.

Today, no oral male contraceptives are available. Existing choices of male contraception include condoms, withdrawal and vasectomy (sterilization).

Unfortunately, condoms and withdrawal have a high rate of user failure. Although vasectomy is an effective contraceptive method, it is a permanent procedure with little chance of reversal which makes it unpopular with some couples.

Hormonal approaches, which are administered by monthly injections, are currently in clinical trials, but these trials have shown an overall failure rate of 6%. With regard to non-hormonal contraceptive compounds, a number of targets have been found, against which such compounds can be directed. These include blocking of the vas deferens; modifying germ-cell adhesion or sperm morphology; affecting Leydig-cell steroidogenesis, modifying semen liquefaction; modifying sperm membrane cholesterol; affecting glycolysis or intracellular pH in sperm; and affecting intercellular bridge formation in germ cells or sperm egg interactions. However, many of these potential targets have associated problems, in particular associated with non-reversible contraceptive action, i.e. they can lead to sterility.

Promising targets for male contraceptives are enzymes of the glycolytic pathway in sperm cells. Targeting the activity of certain enzymes of the sperm cell glycolytic pathway will directly affect their motility and hence male fertility. As sperm cells lack mitochondria, the conversion of pyruvate into lactate by the enzyme lactate dehydrogenase (LDH), is the main producer of ATP. This also means that glycolysis is the major producer of energy supporting sperm cell swimming. LDH comprises a tetrameric structure, built up by combinations of two subunits, LDHA (M, muscle) and LDHB (H, heart). The structural arrangement of these subunits gives rise to five isoforms: the two homotetramers LDH1 (H4, LDHB) found predominantly in the heart and LDH5 (M4, LDHA) which is present in skeletal muscle, as well as three heterotetramers which are found in other tissues (e.g. the lungs and kidneys). The sixth isoform, the homotetramer LDHC (4 subunits of LDHC), is testis- and sperm-specific and is linked to male fertility and is thus the main target for the sperm motility-reducing agents as described herein.

Sperm cells display different patterns of movement that are adapted to their functional needs in space and time. These movements can be monitored, for example, by a Computer Assisted Sperm Analyzer (CASA), which records and analyses the movement of the sperm head. Factors like average path velocity (VAP), curvilinear velocity (VCL), straightness (STR), linearity (LIN) and amplitude of lateral head displacement (ALH) have all been shown to be directly correlated to semen quality.

In semen, sperm cells mostly exhibit a fast forward motion (known as progressive motility) which is necessary for the cells to travel through the cervix. The complex process of sperm capacitation (the penultimate step in the maturation of mammalian spermatozoa which is required to render them competent to fertilize an egg) starts once the cells have entered the uterus. Hyperactivation is a hallmark of capacitation and this motile behavior is observed as a vigorous and asymmetrical swimming pattern that aids the release of spermatozoa from the oviduct epithelium and subsequent penetration of the egg cell. Sperm motility is required for fertilization and compounds which reduce sperm motility can possess contraceptive effects in vitro and in vivo.

For example, Miglustat, marketed by Actelion Pharmaceuticals under the registered trademark Zavesca®, and N-butyldeoxygalactonojirimycin (NB-DGL) are both alkylated imino sugars which have been shown to affect sperm structure and function in mice. Other examples of agents affecting sperm morphology and motility include extracts from Tripterygium wilfordii and Neem tree leaves. Although showing promising results in animal models, none has impaired spermatogenesis in humans.

There remains a need for further male contraceptives, particularly orally-administrable male contraceptives.

We have found that a particular group of piperidine-dione derivatives are effective in inhibiting the sperm-specific enzyme lactate dehydrogenase C (“LDHC”) and thus have potential in directly reducing the motility of sperm. Such compounds are suitable for various in vitro and in vivo uses. They are especially suitable for preventing unwanted fertilization in females, e.g. for use as a male contraceptive, which gives temporary male infertility and which can be administered orally.

Certain piperidine-dione compounds are described by Genentech, Inc. in WO 2015/140133 and in WO 2015/142903. In these earlier applications the compounds are shown to exhibit low LDHA IC₅₀ values in an LDHA enzyme inhibition assay and are proposed for use in the treatment of various cancers and in the control of lactate production in mammalian cell cultures used to produce recombinant proteins. Neither of these documents contains any suggestion that the compounds may have the ability to inhibit LDHC.

### SUMMARY OF THE INVENTION

In one aspect the invention relates to a method reducing sperm motility or of contraception in a subject, said method comprising the step of administering to said subject an effective amount of a compound of formula (I), a stereoisomer, tautomer, pharmaceutically acceptable salt or prodrug thereof:

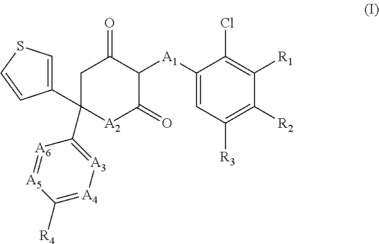

wherein:
A₁ is —O—, —CH₂—, or —S—;
A₂ is NR (wherein R is either H or C₁₋₃ alkyl);
A₃ is N or CR₅;
A₄ is N or CR₆;
A₅ is N or CR₇;
A₆ is N or CR₈;
R₁, R₂ and R₃ are independently selected from H and halogen;
R₄ is selected from:
   H;
   halogen;
   a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
   OR₉ in which R₉ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
   OR₁₀ in which R₁₀ is a C₃₋₈ cycloalkyl group;
R₅ is selected from:
   H;
   hydroxy;
   C₁₋₆ alkyl; and
   C₁₋₆ alkoxy;
R₆ is selected from:
   H;
   halogen;
   C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and aryl groups;
   C₁₋₆ alkoxy optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and C₃₋₈ cycloalkyl groups;
   a 4- to 6-membered heterocyclic ring optionally substituted by one or more substituents selected from the group consisting of halogen, hydroxy, C₁₋₆ alkyl, C₁₋₆ alkoxy, C₁₋₆, haloalkyl, C₁₋₆ hydroxyalkyl, —CO₂H,
   C(O)—O—C₁₋₆ alkyl, —C(O)—C₁₋₆ alkyl, amino, cyano, and nitro groups;
   OR₁₁ in which R₁₁ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
   OR₁₂ in which R₁₂ is a C₃₋₈ cycloalkyl group;
R₇ and R₈ are independently selected from:
   H;
   hydroxy;
   C₁₋₆ alkyl; and
   C₁₋₆ alkoxy;
with the provisos that:
A₃ and A₄ are not both N at the same time; and
A₅ and A₆ are not both N at the same time.

In a further aspect the invention relates to a method of reducing sperm motility or of contraception in a subject, said method comprising the step of administering to said subject an effective amount of a compound of formula (II), a stereoisomer, or pharmaceutically acceptable salt thereof:

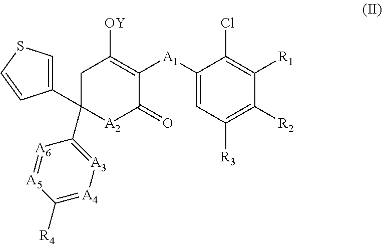

wherein A₁ to A₆ and R₁ to R₄ are as defined herein; and

Y is either H or a progroup as herein defined.

In a further aspect the invention relates to a compound of formula (I) or (II), a stereoisomer, tautomer, pharmaceutically acceptable salt or prodrug thereof, for use in a method of reducing sperm motility or for use as a contraceptive.

A further aspect of the invention relates to the use of a compound of formula (I) or (II), a stereoisomer, a tautomer, pharmaceutically acceptable salt or prodrug thereof, in the manufacture of a medicament for use in a method of reducing sperm motility or for use as a contraceptive.

### DETAILED DESCRIPTION OF THE INVENTION

### Definitions

The term “alkyl” as used herein refers to a monovalent saturated, linear or branched, carbon chain. Examples of alkyl groups include, but are not limited to, methyl, ethyl, n-propyl, iso-propyl, n-butyl, iso-butyl, sec-butyl, tert-butyl, n-pentyl, iso-pentyl, neo-pentyl, n-hexyl, etc. An alkyl group preferably contains from 1-6 carbon atoms, e.g. 1-4 carbon atoms. Unless otherwise specified, any alkyl group may be substituted in one or more positions with a suitable substituent. Where more than one substituent group is present, these may be the same or different. Suitable substituents include hydroxy, C₁₋₆ alkoxy, amino, cyano, and nitro groups, or halogen atoms (e.g. F, Cl or Br).

The term “alkoxy” as used herein refers to an —O-alkyl group, wherein alkyl is as defined herein. Examples of alkoxy groups include, but are not limited to, methoxy, ethoxy, propyloxy, etc. Unless otherwise specified, any alkoxy group may be substituted in one or more positions with a suitable substituent. Where more than one substituent group is present, these may be the same or different. Suitable substituents include hydroxy, C₁₋₆ alkoxy, amino, cyano, and nitro groups, or halogen atoms (e.g. F, Cl or Br).

The term “aryl” as used herein refers to aromatic ring systems. Such ring systems may be monocyclic or bicyclic and contain at least one unsaturated aromatic ring. Where these contain bicyclic rings, these may be fused. Preferably such systems contain from 6-20 carbon atoms, e.g. either 6 or 10 carbon atoms. Examples of such groups include phenyl, 1-napthyl and 2-napthyl. A preferred aryl group is phenyl. Unless stated otherwise, any aryl group may be substituted by one or more substituents as described herein. Where more than one substituent group is present, these may be the same or different. Suitable substituents include hydroxy, C₁₋₆ alkoxy, amino, cyano, and nitro groups, or halogen atoms (e.g. F, Cl or Br).

The term “cycloalkyl” refers to a monovalent, saturated cyclic carbon system. It includes monocyclic and bicyclic rings. Monocyclic rings may contain from 3 to 8 carbon atoms and bicyclic rings may contain from 7 to 14 carbon atoms. Examples of monocyclic cycloalkyl groups include, but are not limited to, cyclopropyl, cyclobutyl, cyclopentyl, cyclohexyl, cycloheptyl, etc. Unless otherwise specified, any cycloalkyl group may be substituted in one or more positions with a suitable substituent. Where more than one substituent group is present, these may be the same or different. Suitable substituents include hydroxy, C₁₋₆ alkoxy, amino, cyano, and nitro groups, or halogen atoms (e.g. F, Cl or Br).

The terms “halogen”, “halo” or “halogen atom” are used interchangeably herein and refer to —F, —Cl, —Br or —I.

The term “haloalkyl” refers to an alkyl group as defined herein in which at least one of the hydrogen atoms of the alkyl group is replaced by a halogen atom, preferably F, Cl or Br. Examples of such groups include —CH₂F, —CHF₂, —CF₃, —CCl₃, —CHCl₂, —CH₂CF₃, etc.

The term “hydroxyalkyl” refers to an alkyl group as defined herein in which at least one of the hydrogen atoms of the alkyl group is replaced by a hydroxy group. Examples of such groups include methyl, ethyl, n-propyl, iso-propyl, n-butyl, iso-butyl, sec-butyl, tert-butyl, n-pentyl, iso-pentyl, neo-pentyl, n-hexyl, etc. in which one or more hydrogen atoms are replaced by —OH.

The term “heterocyclic ring” as used herein refers to a saturated or partially unsaturated, 4- to 6-membered (preferably 5- or 6-membered) carbocyclic system in which at least one ring atom is a heteroatom selected from nitrogen, oxygen and sulfur, the remaining ring atoms being carbon. The heterocyclic ring structure may be linked to the remainder of the molecule through a carbon atom or through a nitrogen atom. Examples of heterocyclic rings include, but are not limited to, tetrahydrofuran, piperidine, pyrrolidine, dioxane, pyrene, morpholine, etc. Unless otherwise stated, any heterocyclic ring mentioned herein may optionally be substituted by one or more groups, which may be identical or different, for example hydroxy, C₁₋₆ alkyl, C₁₋₆ alkoxy, amino, cyano, and nitro groups, or halogen atoms (e.g. F, Cl or Br).

The compounds herein described may contain one or more stereocenters and may therefore exist in different stereoisomeric forms. The term “stereoisomer” refers to compounds which have identical chemical constitution but which differ in respect of the spatial arrangement of the atoms or groups. Examples of stereoisomers are enantiomers and diastereomers. The term “enantiomers” refers to two stereoisomers of a compound which are non-superimposable mirror images of one another. The term “diastereoisomers” refers to stereoisomers with two or more stereocenters which are not mirror images of one another. The invention is considered to extend to the use of diastereomers and enantiomers, as well as racemic mixtures and enantioenriched mixtures in which the ratio of the enantiomers is other than 1:1.

The compounds herein described may be resolved into their enantiomers and/or diastereomers. For example, where these contain only one chiral center, these may be provided in the form of a racemate or racemic mixture (a 50:50 mixture of enantiomers) or may be provided as pure enantiomers, i.e. in the R- or S-form. Any of the compounds which occur as racemates may be separated into their enantiomers by methods known in the art, such as column separation on chiral phases or by recrystallization from an optically active solvent. Those compounds with at least two asymmetric carbon atoms may be resolved into their diastereomers on the basis of their physical-chemical differences using methods known per se, e.g. by chromatography and/or fractional crystallization, and where these compounds are obtained in racemic form, they may subsequently be resolved into their enantiomers.

The term “tautomer” as used herein refers to structural isomers which readily interconvert by way of a chemical reaction which may involve the migration of a proton accompanied by a switch of a single bond and adjacent double bond. It includes, in particular, keto-enol tautomers. Dependent on the conditions, the compounds may predominantly exist either in the keto or enol form and the invention is not intended to be limited to the particular form shown in any of the structural formulae given herein.

The term “pharmaceutically acceptable salt” as used herein refers to any pharmaceutically acceptable organic or inorganic salt of any of the compounds herein described. A pharmaceutically acceptable salt may include one or more additional molecules such as counter-ions. The counter-ions may be any organic or inorganic group which stabilizes the charge on the parent compound. If the compound for use in the invention is a base, a suitable pharmaceutically acceptable salt may be prepared by reaction of the free base with an organic or inorganic acid. If the compound for use in the invention is an acid, a suitable pharmaceutically acceptable salt may be prepared by reaction of the free acid with an organic or inorganic base. Non-limiting examples of suitable salts are described herein.

The term “pharmaceutically acceptable” means that the compound or composition is chemically and/or toxicologically compatible with other components of the formulation or with the patient (e.g. human) to be treated.

By “a pharmaceutical composition” is meant a composition in any form suitable to be used for a medical purpose.

The term “prodrug” refers to a derivative of an active compound which undergoes a transformation under the conditions of use, for example within the body, to release an active drug. A prodrug may, but need not necessarily, be pharmacologically inactive until converted into the active drug. As used herein, the term “prodrug” extends to any compound which under physiological conditions is converted into any of the active compounds herein described. Suitable prodrugs include compounds which are hydrolyzed under physiological conditions to the desired molecule.

Prodrugs may typically be obtained by masking one or more functional groups in the parent molecule which are considered to be, at least in part, required for activity using a progroup. By “progroup” as used herein is meant a group which is used to mask a functional group within an active drug and which undergoes a transformation, such as cleavage, under the specified conditions of use (e.g. administration to the body) to release a functional group and hence provide the active drug. Progroups are typically linked to the functional group of the active drug via a bond or bonds that are cleavable under the conditions of use, e.g. in vivo. Cleavage of the progroup may occur spontaneously under the conditions of use, for example by way of hydrolysis, or it may be catalyzed or induced by other physical or chemical means, e.g. by an enzyme, by exposure to light, by exposure to a change in temperature, or to a change in pH, etc. Where cleavage is induced by other physical or chemical means, these may be endogenous to the conditions of use, for example pH conditions at a target site, or these may be supplied exogenously.

As used herein, “treatment” includes any therapeutic application that can benefit a human or non-human animal (e.g. a non-human mammal). Both human and veterinary treatments are within the scope of the present invention, although primarily the invention is aimed at the treatment of humans. Treatment may be in respect of an existing condition or it may be prophylactic.

As used herein, a “pharmaceutically effective amount” relates to an amount that will lead to the desired pharmacological and/or therapeutic effect, i.e. an amount of the agent which is effective to achieve its intended purpose. While individual patient needs may vary, determination of optimal ranges for effective amounts of the active agent is within the capability of one skilled in the art. Generally, the dosage regimen for treating a condition with any of the compounds described herein is selected in accordance with a variety of factors including the nature of the medical condition and its severity.

As used herein, “lactate dehydrogenase C” or “LDHC” refers to an enzyme that is predominantly expressed in the testes and sperm cells and which converts pyruvate originating from glycolysis to lactate, coupled with oxidation of NADH to NAD+.

The invention is based, at least in part, on the finding that certain piperidine-dione compounds as herein defined have LDHC inhibitory activity. This discovery leads to the use of the compounds to reduce sperm motility or in a method of contraception in subjects, e.g. in humans.

The compounds for use according to the invention are those of formula (I), their stereoisomers, tautomers, pharmaceutically acceptable salts, and prodrugs:

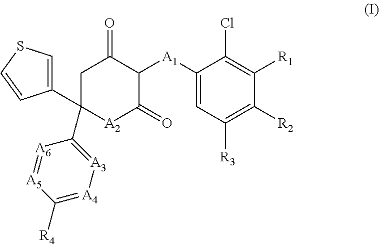

wherein:

A₁ is —O—, —CH₂—, or —S—;

A₂ is NR (wherein R is either H or C₁₋₃ alkyl);

A₃ is N or CR₅;

A₄ is N or CR₆;

A₅ is N or CR₇;

A₆ is N or CR₈;

R₁, R₂ and R₃ are independently selected from H and halogen;

R₄ is selected from:
H;
halogen;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
OR₉ in which R₉ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₁₀ in which R₁₀ is a C₃₋₈ cycloalkyl group;

R₅ is selected from:
H;
hydroxy;
C₁₋₆ alkyl; and
C₁₋₆ alkoxy;

R₆ is selected from:
H;
halogen;
C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, nitro and aryl groups (e.g. C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, and nitro groups);
C₁₋₆ alkoxy optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and C₃₋₈ cycloalkyl groups;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H,
—C(O)—O—C₁₋₆ alkyl, —C(O)—C₁₋₆ alkyl, amino, cyano, and nitro groups;
OR₁₁ in which R₁₁ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₁₂ in which R₁₂ is a C₃₋₈ cycloalkyl group;

R₇ and R₈ are independently selected from:
H;
hydroxy;
C₁₋₆ alkyl; and
C₁₋₆ alkoxy;

with the provisos that:

A₃ and A₄ are not both N at the same time; and

A₅ and A₆ are not both N at the same time.

In an embodiment A₁ is —S—.

In an embodiment A₂ is NH or N-methyl, preferably NH.

In an embodiment, A₄ is CR₆.

In an embodiment, A₅ is CR₇.

In an embodiment, A₆ is CR₈.

In an embodiment A₃ is N. In one embodiment, when A₃ is N, A₄ is other than CH and/or R₄ is H.

Those compounds of formula (I) in which A₃ is N, A₄ is CR₆, and A₅ and A₆ are both CH represent a preferred embodiment of the compounds for use in the invention and may be represented by formula (III):

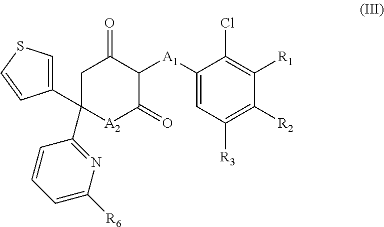

wherein A₁, A₂, R₁ to R₃ and R₆ are as herein defined.

In the compounds of formula (III), R₆ may be other than H. For example, R₆ may be selected from any of the following:
halogen;
C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and aryl groups (e.g. C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, and nitro groups);
C₁₋₆ alkoxy optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and C₃₋₈ cycloalkyl groups;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
OR₁₁ in which R₁₁ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₁₂ in which R₁₂ is a C₃₋₈ cycloalkyl group.

In an embodiment of formula (III), R₆ may be halogen (e.g. Br or Cl, preferably Br), or it may be an optionally substituted C₁₋₆ alkoxy group.

In an embodiment, R₆ is an optionally substituted C₁₋₆ alkoxy group as herein defined. Where this is substituted, suitable substitutents include C₃₋₈ cycloalkyl groups, e.g. unsubstituted cyclopentyl.

In an embodiment, R₆ is a C₁₋₆ alkyl substituted by an optionally substituted aryl group, e.g. C₁-alkyl substituted by an optionally substituted aryl group. Where the aryl group is substituted, suitable substituents may include one or more halogen atoms, e.g. selected from F, Cl and Br, preferably F. In this embodiment, the aryl group may be optionally substituted phenyl.

In one embodiment A₃ is CH.

In one embodiment, when A₃ is CH, R₄ is other than H, for example R₄ is an optionally substituted 4- to 6-membered heterocyclic ring.

Compounds of formula (I) in which A₃ is CH, R₄ is other than H, and each of A₄, A₅ and A₆ is CH represent an embodiment of the compounds for use in the invention and may be represented by formula (IV):

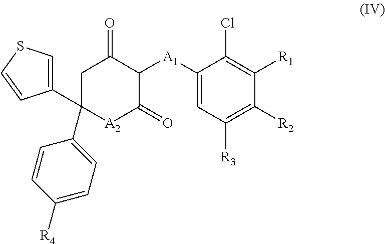

wherein A₁, A₂, R₁ to R₃ are as herein defined; and

R₄ is selected from any of the following:
halogen;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
   OR₉ in which R₉ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
   OR₁₀ in which R₁₀ is a C₃₋₈ cycloalkyl group;

In any of the compounds herein described, R₁, R₂ and R₃ are each independently selected from H and halogen.

In one embodiment, R₁, R₂ and R₃ are each H.

In certain embodiments of the invention at least one of R₁, R₂ and R₃ is halogen. In one embodiment, one or two of R₁, R₂ and R₃ are halogen. In one embodiment, one of R₁, R₂ and R₃ is halogen. In another embodiment, R₁ is halogen and R₂ and R₃ are H. In another embodiment, R₂ is halogen and R₁ and R₃ are H. In another embodiment R₃ is halogen and R₁ and R₂ are H.

Where one or more of R₁, R₂ and R₃ is halogen, these may independently be selected from —F, —Cl and —Br. In one embodiment these are selected from —F and —Cl.

In one embodiment the compounds of the invention are those in which R₁ or R₃ is —Cl, or in which R₂ is —F.

Examples of compounds for use in accordance with the invention include, but are not limited to, the following:
6-[6-(cyclopentylmethoxy)pyridin-2-yl]-3-[(2,4-dichlorophenyl)sulfanyl]-6-(thiophen-3-yl)piperidine-2,4-dione;
3-[(2-chloro-4-fluorophenyl)sulfanyl]-6-[6-(cyclopentylmethoxy)pyridin-2-yl]-6-(thiophen-3-yl)piperidine-2,4-dione;
6-[6-(cyclopentylmethoxy)pyridin-2-yl]-3-[(2,5-dichlorophenyl)sulfanyl]-6-(thiophen-3-yl)piperidine-2,4-dione;
6-[6-(cyclopentylmethoxy)pyridin-2-yl]-3-[(2,3-dichlorophenyl)sulfanyl]-6-(thiophen-3-yl)piperidine-2,4-dione;
3-((2-chloro-4-fluorophenyl)thio)-6-(4-morpholinophenyl)-6-(thiophen-3-yl)piperidine-2,4-dione;
3-((2,5-dichlorophenyl)thio)-6-(4-morpholinophenyl)-6-(thiophen-3-yl)piperidine-2,4-dione;
3-((2-chlorophenyl)thio)-6-(4-morpholinophenyl)-6-(thiophen-3-yl)piperidine-2,4-dione;
3-((2-chlorophenyl)thio)-6-(6-(cyclopentylmethoxy)pyridin-2-yl)-6-(thiophen-3-yl)piperidine-2,4-dione;
5-((2,5-dichlorophenyl)thio)-2-(4-morpholinophenyl)-6-oxo-2-(thiophen-3-yl)-1,2,3,6-tetrahydropyridin-4-yl isonicotinate;
3-((2-chlorophenyl)thio)-6-(6-(oxetan-3-yloxy)pyridin-2-yl)-6-(thiophen-3-yl)piperidine-2,4-dione;
3-((2-chlorophenyl)thio)-6-(6-(3-fluorobenzyl)pyridin-2-yl)-6-(thiophen-3-yl)piperidine-2,4-dione;
3-((2-chlorophenyl)thio)-6-(6-(isopentyloxy)pyridin-2-yl)-6-(thiophen-3-yl)piperidine-2,4-dione;

and their stereoisomers, tautomers, pharmaceutically acceptable salts, and prodrugs thereof.

The compounds for use according to the invention may be converted into a salt thereof, particularly into a pharmaceutically acceptable salt thereof with an inorganic or organic acid or base. Acids which may be used for this purpose include hydrochloric acid, hydrobromic acid, sulfuric acid, sulfonic acid, methanesulfonic acid, phosphoric acid, fumaric acid, succinic acid, lactic acid, citric acid, tartaric acid, maleic acid, acetic acid, trifluoroacetic acid and ascorbic acid. Bases which may be suitable for this purpose include alkali and alkaline earth metal hydroxides, e.g. sodium hydroxide, potassium hydroxide or cesium hydroxide, ammonia and organic amines such as diethylamine, triethylamine, ethanolamine, diethanolamine, cyclohexylamine and dicyclohexylamine. Procedures for salt formation are conventional in the art.

The compounds for use in the invention may be provided in the form of the corresponding enol derivatives. These include compounds represented by formula (IIa), their stereoisomers, pharmaceutically acceptable salts and prodrugs:

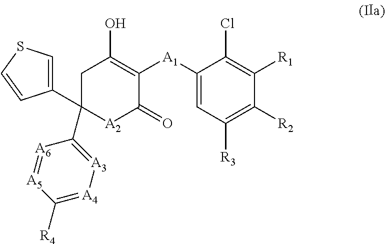

wherein A₁ to A₆ and R₁ to R₄ are as defined herein. Any of these groups may be defined in accordance with any of the embodiments herein described with respect to the compounds of formula (I).

Any of the compounds for use in the invention may alternatively be provided in the form of a prodrug. Prodrugs may be obtained by masking one or more functional groups in the parent molecule using a progroup as herein defined. A wide variety of progroups suitable for masking functional groups in active compounds to provide prodrugs are well known in the art. For example, a hydroxy functional group may be masked as an ester (e.g. an alkyl, aryl or heteroaryl ester), a phosphate ester, or a sulfonate ester which may be hydrolyzed in vivo to provide the parent hydroxy group. An amide functional group may be hydrolyzed in vivo to provide the parent amino group. A carboxyl group may be masked as an ester or amide which may be hydrolyzed in vivo to provide the parent carboxyl group. Other examples of suitable progroups will be apparent to those of skill in the art.

In one embodiment, the compounds of the invention have a hydroxy functional group that can be derivatized to produce suitable prodrugs. For example, the hydroxy group can be converted to an alkyl, aryl or heteroaryl ester, a phosphate ester, a sulfonate ester, etc.

In one embodiment, the prodrugs for use in accordance with the invention are those of formula (IIb), their stereoisomers, or pharmaceutically acceptable salts:

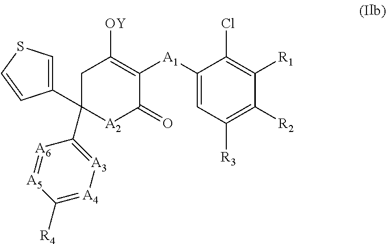

wherein A₁ to A₆ and R₁ to R₄ are as defined herein; and

Y represents a progroup.

Any of A₁ to A₆ and R₁ to R₄ in formula (IIb) may be defined in accordance with any of the embodiments herein described with respect to the compounds of formula (I).

Suitable progroups, Y, include those which together with the —O— atom to which they are linked form an alkyl ester, aryl ester, heteroaryl ester, phosphate ester, or sulfonate ester group. The precise nature of the progroup, Y, may be selected according to need, for example depending on the desired oil or water solubility of the prodrug, its intended mode of administration and/or its intended mode of metabolism at the target site to produce the active drug compound. The progroup may, for example, be hydrophilic or lipophilic in order to increase or decrease water solubility as required. The choice of progroup may also impart other desirable properties such as enhanced absorption from the gastrointestinal tract, improved drug stability, etc.

As will be understood, the compounds described herein may exist in various stereoisomeric forms, including enantiomers, diastereomers, and mixtures thereof. The invention encompasses all optical isomers of the compounds described herein and mixtures of optical isomers. Hence, compounds that exist as diastereomers, racemates and/or enantiomers are within the scope of the invention.

In one embodiment, the invention provides compounds having the following stereochemistry, their tautomers, pharmaceutically acceptable salts, and prodrugs:

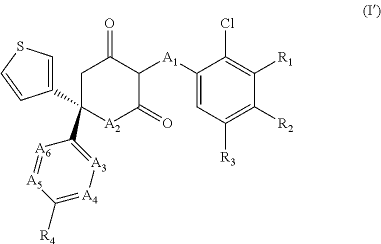

wherein A₁ to A₆ and R₁ to R₄ are as herein defined.

In another embodiment, the invention provides compounds having the following stereochemistry, their tautomers, pharmaceutically acceptable salts, and prodrugs:

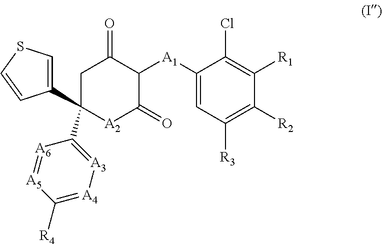

wherein A₁ to A₆ and R₁ to R₄ are as herein defined.

Any of compounds (II), (IIa), (IIb), (III) and (IV) having this stereochemistry and, where appropriate, any tautomer, pharmaceutically acceptable salt, or prodrug thereof form further embodiments of the invention.

The compounds for use according to the invention may be prepared from readily available starting materials using synthetic methods known in the art, for example, using methods analogous to those described in WO 2015/140133, the entire content of which is incorporated herein by reference. In some cases, the compounds may be known in the art and may be prepared using methods previously described, such as in WO 2015/140133.

The following schemes show general methods for preparing the compounds of formula (I) and key intermediates. The compounds used as starting materials are either known from the literature or may be commercially available. Alternatively, these may readily be obtained by methods known from the literature. As will be understood, other synthetic routes may be used to prepare the compounds using different starting materials, different reagents and/or different reaction conditions. A more detailed description of how to prepare the compounds in accordance with the invention is found in the Examples.

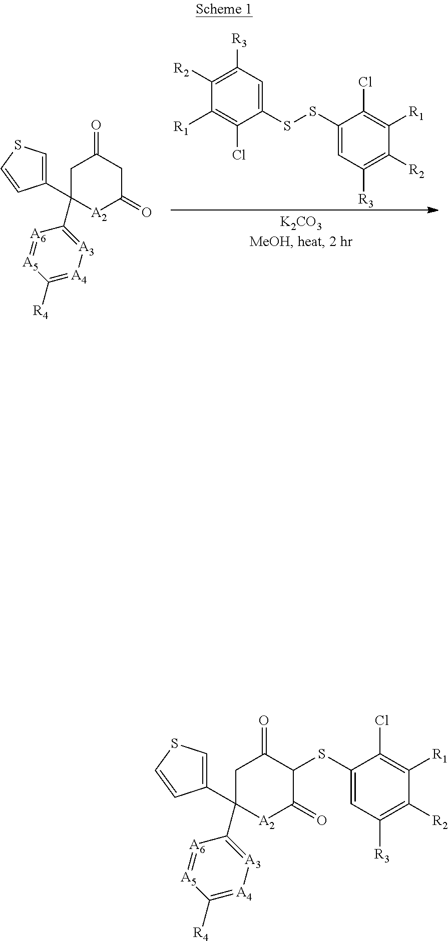

In scheme 1, A₂ is NR (wherein R is H or C₁₋₃ alkyl), and A₃ to A₆, R₁ to R₄ are as herein defined.

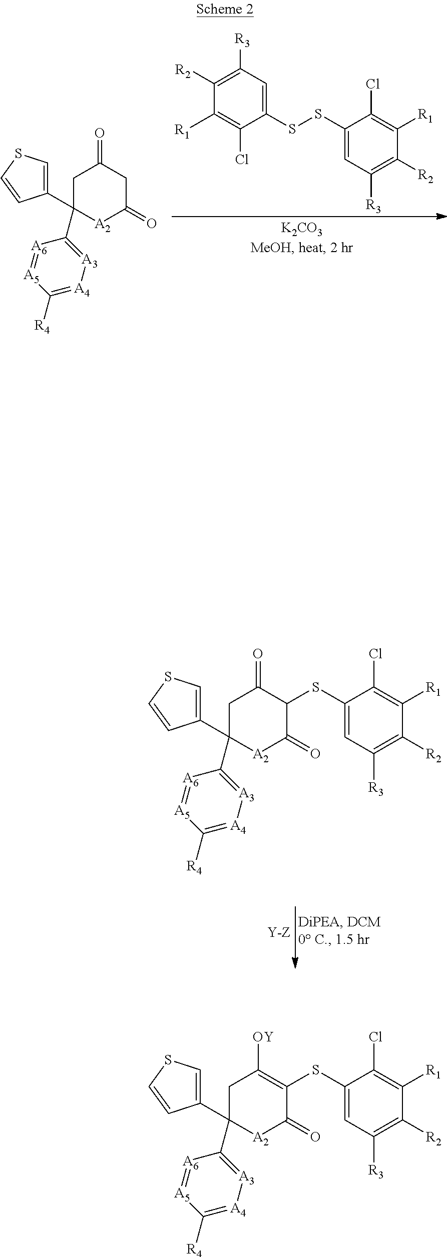

In scheme 2, A₂ to A₆, R₁ to R₄ and Y are as herein defined, and Z is a leaving group such as a halogen atom, e.g. Cl.

The compounds herein described have valuable pharmacological properties, particularly an inhibitory effect on LDHC. LDHC plays a central role in the energy metabolism in sperm cells and thus the compounds have a motility-reducing effect on sperm. In view of their ability to reduce sperm motility, the compounds herein described are suitable for contraceptive uses in vivo.

For use in a therapeutic or prophylactic treatment, the compounds herein described will typically be formulated as a pharmaceutical formulation together with one or more pharmaceutically acceptable carriers, excipients or diluents. Acceptable carriers, excipients and diluents for therapeutic use are well known in the art and can be selected with regard to the intended route of administration and standard pharmaceutical practice. Examples include binders, lubricants, suspending agents, coating agents, solubilizing agents, preserving agents, wetting agents, emulsifiers, surfactants, sweeteners, colorants, flavoring agents, antioxidants, odorants, buffers, stabilizing agents and/or salts.

The compounds for use in the invention may be formulated with one or more conventional carriers and/or excipients according to techniques well known in the art. Typically, the compositions will be adapted for oral or parenteral administration, for example by intradermal, subcutaneous, intraperitoneal or intravenous injection.

For example, these may be formulated in conventional oral administration forms, e.g. tablets, coated tablets, capsules, powders, granulates, solutions, dispersions, suspensions, syrups, emulsions, etc. using conventional excipients, e.g. solvents, diluents, binders, sweeteners, aromas, pH modifiers, viscosity modifiers, antioxidants, etc. Suitable excipients may include, for example, corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose or fatty substances such as saturated fats or suitable mixtures thereof, etc.

Where parenteral administration is employed this may for example be by means of intradermal, subcutaneous, intraperitoneal, intravenous, intramuscular or intratesticular injection. For this purpose, sterile solutions containing the active agent may be employed, such as an oil-in-water emulsion. Where water is present, an appropriate buffer system (e.g., sodium phosphate, sodium acetate or sodium borate) may be added to prevent pH drift under storage conditions.

Alternatively, the compounds herein described may be formulated for topical administration, e.g. in the form of a gel, cream, emulsion, paste, etc., e.g. comprising a compound as herein described along with a conventional carrier or excipient. In one embodiment, the compounds herein described may be formulated as a gel, especially a vaginal gel, which may optionally contain further contraceptive and/or spermicidal agents. In another embodiment, the compounds herein described may be formulated as a gel or cream for topical is a patch formulation, for example a composition comprising a compound as herein described disposed on and/or embedded in a structural layer such as a backing material. In other embodiments, the compounds herein described may be formulated for transdermal administration.

The use of orally administrable compositions, e.g. tablets, coated tablets, capsules, syrups, etc. is especially preferred.

The formulations may be prepared using conventional techniques, such as dissolution and/or mixing procedures.

In one aspect the compounds herein described are used in maintenance of the life and health of the individual treated with the compound or of an individual who will come into contact with sperm treated with the compound. For example, in cases where pregnancy represents a particular risk to a female (i.e. a risk higher than that associated with pregnancy in a healthy female), treatment of the female, or of a male partner thereof, with a compound as herein described may be considered prophylactic of the condition leading to the risk associated with pregnancy.

In one embodiment, the compounds herein described may be used in reducing the fertility of a male subject or for reducing sperm motility in a male subject or for use as a male contraceptive. In a related aspect, the invention provides compounds and compositions as defined herein for administration to a female subject of fertile age (e.g. shortly before, during and/or shortly after intercourse) for reducing the likelihood of conception in said female subject.

In a related aspect, the invention provides compounds and compositions as defined herein in the preparation of a medicament for reducing the fertility of a male subject or for reducing sperm motility in a male subject or for use as a male contraceptive. In a related aspect, the invention provides compounds and compositions as defined herein in the preparation of a medicament for administration to a female subject of fertile age (e.g. shortly before, during and/or shortly after intercourse) for reducing the likelihood of conception in said female subject.

In a related aspect, the invention provides a contraceptive method, especially a method of reducing the fertility of a male subject, especially a male subject having reached sexual maturity (e.g. post-puberty in humans), comprising administering an effective amount of a compound or composition as defined herein to said subject.

Also provided is a method of reducing sperm motility in a male subject, especially a subject having reached sexual maturity (e.g. post-puberty in humans), comprising administering an effective amount of a compound or composition as defined herein to said subject.

The invention further provides a method of contraception in a male subject, especially a subject having reached sexual maturity (e.g. post-puberty in humans), comprising administering an effective amount of a compound or composition as defined herein to said subject.

In a related aspect the invention further provides a method of contraception in a female subject, especially a female subject of fertile age (e.g. post-puberty and pre-menopause in humans), comprising administering an effective amount of a compound or composition as defined herein to said subject.

As used herein, the term “effective amount” is intended to mean a contraceptively-effective amount or a sperm motility-reducing amount.

The dosage required to achieve the desired activity will depend on the compound which is to be administered, the patient and the method and frequency of administration and may be varied or adjusted according to choice. The appropriate dosage could be determined by one of skill in the art. Typically, the dosage may be expected to be in the range from 1 μg to 50 mg per kg bodyweight, preferably 0.01 to 10 mg per kg bodyweight, especially from 0.1 to 5 mg per kg bodyweight. Administration of the compounds of the invention may be long-term (e.g. daily for a period of at least one week, e.g. two weeks, one month, two months, six months or more than six months), especially where the subject is a male subject, or may be short term, especially where the subject is a female, e.g. before intercourse.

Administration may be via any conventional route, for example in solid, gel, cream or emulsion form for oral, sub-lingual, nasal, rectal, topical or vaginal application; by inhalable powder or liquid form for inhalation; or by solid or liquid form for injection (e.g. intravenous, intra-muscular or intra-testicular injection). Preferred forms of administration to male subjects are topical, especially to the skin of the male genitals, oral and by injection, especially intravenous injection. Oral administration is an especially preferred route of administration for male subjects.

Preferred forms of administration to female subjects are vaginal, e.g. in the form of a gel, cream or emulsion, oral and by injection, especially intravenous injection. Vaginal administration is an especially preferred route of administration for female subjects.

Preferred subjects according to the invention are mammalian subjects, especially domestic animals (e.g. dogs, cats etc.), farm animals (e.g. sheep, cows, pigs etc.) and laboratory animals (e.g. mice, rats, monkeys etc.). Especially preferred subjects are human subjects, especially for the aspects of the invention related to contraception in cases where there are no particular (i.e. abnormal) medical risks associated with unplanned pregnancy. The compounds herein described are most preferably for administration to male human subjects. In some embodiments of the invention, the subject is a fertile (i.e. sperm-producing) mammal, preferably a fertile male human.

As used herein, the term “reducing sperm motility” is intended to mean reducing sperm motility compared to the motility of sperm from a control subject (or from the same subject) to whom the compound or composition of the invention has not been administered. Compounds of the invention are capable of reducing all forms of sperm motility, e.g. progressive motility and hyperactive motility. Progressive sperm may, for example, be defined as those cells demonstrating an average path velocity (VAP) of greater than 25 μm/s and a straightness (STR) of greater than 80%. Hyperactive motility may, for example, be defined according to the parameters discussed in Kay et al. (Human Reproduction Update, 1998, Vol. 4, No. 6, pp. 776-786). In particular, hyperactive sperm may be defined as those cells demonstrating a curvilinear velocity (VCL) of greater than 90 μm/s and a linearity (LIN) of less than 20% along with a dancemean (the mean lateral head displacement divided by the linearity) of greater than 45.8 μm/s. Alternatively, hyperactive sperm may be defined as those cells demonstrating a VCL of greater than 91.5 μm/s and a LIN of less than 33.1% along with an amplitude of lateral head displacement (ALH) of more than 9.9 μm, a dancemean of more than 35.2 μm/s and a straight line velocity (VSL) of less than 46.4 μm/s.

In a preferred embodiment, the compounds and compositions for use in the invention are capable of reducing the proportion of progressive and/or hyperactive mature sperm cells in a male subject to whom the compound is to be administered (or in a female subject administered said compound who is or will be exposed to said sperm cells) by at least 50%, preferably by at least 80% or at least 90%, especially preferably by at least 95% or 98%.

In a further embodiment, the invention provides a compound or composition as defined herein in combination with instructions or directions for use, e.g. for reducing sperm motility in a male subject or for use as a male or female contraceptive.

The pharmacological properties of the compounds of the invention can be analyzed using standard assays for functional activity. Detailed protocols for testing of the compounds of the invention are provided in the Examples.

### EXAMPLES

The invention will now be described in more detail by way of the following non-limiting Examples and with reference to the accompanying FIGURE, in which:

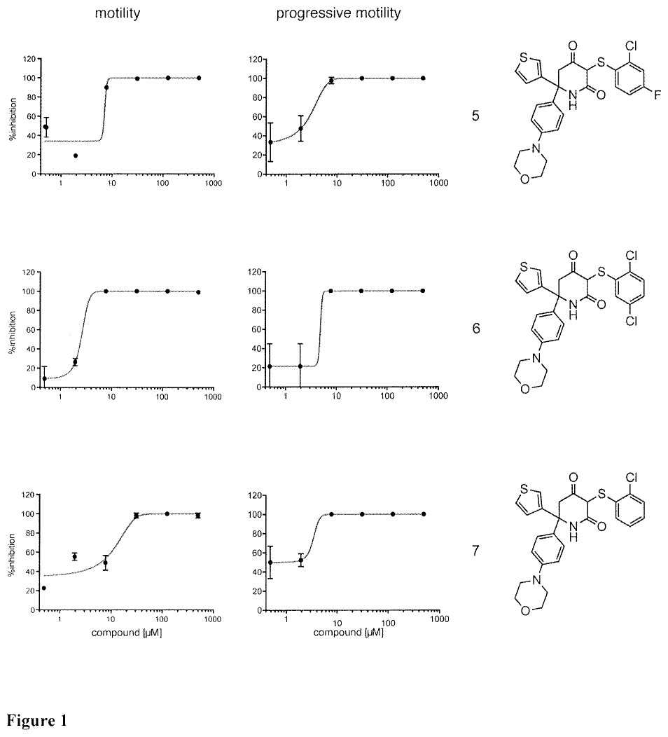

FIG. 1 shows potency (IC₅₀) values calculated for the compounds of Examples 5, 6 and 7 based on a concentration-dependent reduction in sperm motility.

The chemical reactions described in the Examples may readily be adapted to prepare other LDHC inhibitors for use in accordance with the invention, for example by using other reagents known in the art, by modifying the reaction conditions, and/or by choosing any suitable protecting groups, etc.

All reagents and solvents commercially available were used without further purifications. NMR (¹H, ¹³C) spectra were recorded on a Bruker AVII-400 MHz, AVIII-400 MHz or a DPX-300 MHz spectrometer. Coupling constants (J) are reported in hertz (Hz), and chemical shifts are reported in parts per million (ppm) relative to CDCl₃ (7.26 ppm for ¹H and 77.16 ppm for ¹³C), methanol-d₄ (3.31 ppm for ¹H and 49.15 ppm for ¹³C) and DMSO-d₆ (2.50 ppm for ¹H and 39.52 ppm for ¹³C). All yields are uncorrected.

### Abbreviations

DCM: dichloromethane; hr: hour; MeOH: methanol; THF: tetrahydrofuran; e.e.: enantiomeric excess; Rf: retention time.

### Preparation of Starting Materials

### A. Preparation of 6-(6-bromopyridin-2-yl)-6-(thiophen-3-yl)piperidine-2,4-dione

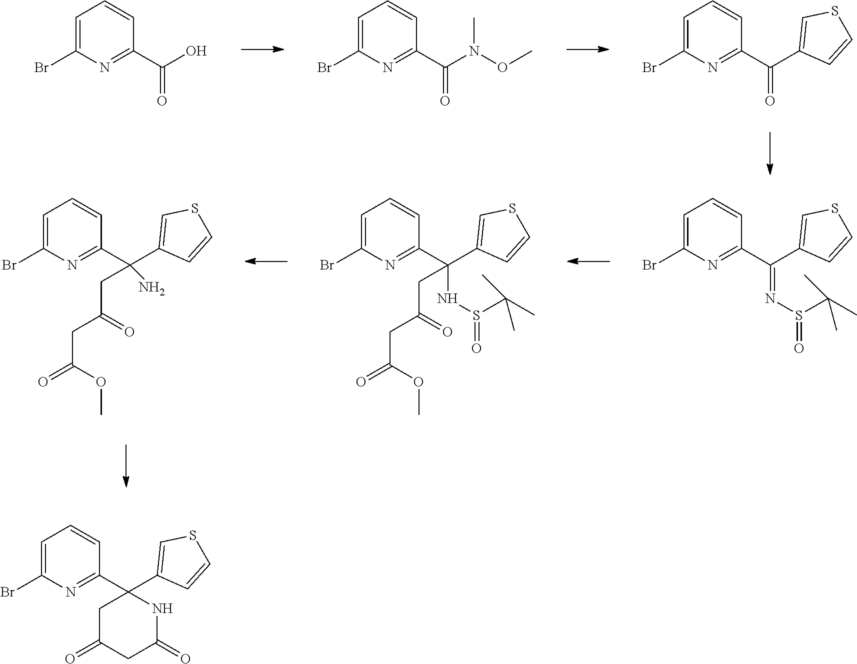

6-(6-Bromopyridin-2-yl)-6-(thiophen-3-yl)piperidine-2,4-dione was prepared using the procedure described in WO 2015/140133, or suitably modified versions thereof.

Step A: N,O-dimethylhydroxylamine hydrochloride (14.6 g, 0.15 mol), HATU (57.0 g, 0.15 mol) and diisopropylethylamine (47.8 g, 0.37 mol) were added to a slurry of 6-bromopicolinic acid (25.3 g, 0.125 mol) in DCM (370 mL). The mixture was stirred at room temperature for 3 hr. The reaction mixture was washed with aqueous HCl 1M (2×200 mL) and formed white solids were filtered off. After concentration under reduced pressure, the crude product was purified by Kugelrohr distillation and silica gel chromatography (hexanes/ethyl acetate: 10 to 25%) to give 6-bromo-N-methoxy-N-methylpicolinamide in 74% yield.

Step B: n-Butyllithium (48 mL, 0.12 mol) was slowly added to a solution of 3-bromothiophene (19.6 g, 0.12 mol) in di-isopropyl ether (280 mL) at −78° C. After stirring at −78° C. for 30 min, 6-bromo-N-methoxy-N-methylpicolinamide (22.5 g, 92 mmol) in di-isopropylether (30 mL) was slowly added and the mixture was stirred at −78° C. for 2 hr. The reaction mixture was quenched with aqueous saturated NH₄Cl (85 mL), then warmed to ambient temperature. The solution was diluted with ethyl acetate (110 mL), washed with water (3×100 mL) and brine (50 mL), dried over Na₂SO₄ and concentrated under reduced pressure to give (6-bromopyridin-2-yl)(thiophen-3-yl)methanone in 56% yield.

Step C: (6-Bromopyridin-2-yl)(thiophen-3-yl)methanone (13.8 g, 51.5 mmol) and titanium ethoxide (31.4 mL, 150 mmol) were added to a solution of 2-methylpropane-2-sulfinamide (12.2 g, 100 mmol) in THF (200 mL). The mixture was stirred under reflux for 20 hr. The solution was allowed to cool to ambient temperature and poured into ice water, filtered, and washed with ethyl acetate (5×100 mL). The filtrate was extracted with ethyl acetate (2×50 mL), and the combined organic phases were washed with brine (50 mL), dried over Na₂SO₄ and concentrated under reduced pressure. The crude product was purified by flash chromatography (SiO₂, hexanes/ethyl acetate: 10 to 25%) to give N-((6-bromopyridin-2-yl)(thiophen-3-yl)methylene)-2-methylpropane-2-sulfinamide in 88% yield.

Step D: Methyl 3-oxobutanoate (10.5 g, 90 mmol,) in THF (20 mL) was added to a suspension of NaH (3.6 g, 90 mmol,) in THF (200 mL) at 0° C. n-Butyllithium (36 mL, 90 mmol) was slowly added to the mixture and the reaction was stirred at 0° C. for 30 min. N-((6-bromopyridin-2-yl)(thiophen-3-yl)methylene)-2-methylpropane-2-sulfinamide (16.4 g, 45 mmol,) in THF (50 mL) was added to the mixture and stirred at 0° C. for another 2 hr. The mixture was allowed to warm to room temperature overnight and cooled to 0° C. The reaction was quenched with saturated NH₄Cl (100 mL) and diluted with ethyl acetate (85 mL). The organic phase was washed with water (2×100 mL), dried over anhydrous Na₂SO₄, filtered and concentrated to give methyl 5-(6-bromopyridin-2-yl)-5-((tert-butylsulfinyl)amino)-3-oxo-5-(thiophen-3-yl)pentanoate.

Step E: TMSCl (19.1 g, 0.18 mol) was slowly added to methanol (100 mL) and added to a solution of methyl 5-(6-bromopyridin-2-yl)-5-((tert-butylsulfinyl)amino)-3-oxo-5-(thiophen-3-yl)pentanoate (45 mmol) in MeOH (200 mL) at 0° C. The mixture was stirred at room temperature for 1 hr, then cooled to 0° C. and slowly acidified to pH 7 using aqueous NaOH 2M (80 mL). The solvent was removed under reduced pressure.

The crude product was extracted with ethyl acetate (2×100 mL), and the combined organic phases were washed with brine (50 mL), dried over Na₂SO₄, filtered and concentrated to give methyl 5-amino-5-(6-bromopyridin-2-yl)-3-oxo-5-(thiophen-3-yl)pentanoate.

Step F: Potassium carbonate (20.7 g, 150 mmol) was added to a solution of methyl 5-amino-5-(6-bromopyridin-2-yl)-3-oxo-5-(thiophen-3-yl)pentanoate (45 mmol) in MeOH (150 mL). The mixture was stirred under reflux for 2 hr and overnight at room temperature. Methanol was removed under reduced pressure, the crude product was dissolved in water (100 mL), and washed with ethyl acetate (2×40 mL). The aqueous layer was acidified to pH 4 using aqueous HCl 3N (95 mL). The aqueous phase was extracted with ethyl acetate (5×40 mL). The combined organic phases were dried over anhydrous MgSO₄, filtered and concentrated to give 6-(6-bromo-2-pyridinyl)-6-(3-thienyl)piperidine-2,4-dione in 41% yield over 3 steps.

### B. Preparation of Disulfides

Method A: The phenyl sulfide (6.2 mmol, 1 eq) was dissolved in DCM (1 mL). CF₃CH₂OH (3 mL) and H₂O₂ solution (0.66 mL, 6.8 mmol, 1.1 eq) was added. The reaction mixture was stirred at room temperature overnight under vigorous stirring.

The white precipitate was filtered and dried under reduced pressure to deliver the desired disulfide.

Method B: The phenyl sulfide (10 mmol, 1 eq) was dissolved in CHCl₃ (50 mL) and 1,3-dibromo-5,5-dimethylhydantoin (1.43 g, 5 mmol, 0.5 eq.) was added. After 1 hr at room temperature, a saturated aqueous solution of sodium thiosulfate was added=10 mL). The phases were separated and the aqueous phase was extracted with DCM (2×20 mL). The combined organic phases were dried with Na₂SO₄, filtered and concentrated under reduced pressure. The crude product was purified by flash chromatography on silica gel (heptane/ethyl acetate: 95/5) to deliver the desired disulfide.

### Preparation of 1,2-bis(2-chlorophenyl)disulfane

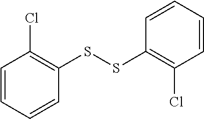 [SMILES: Clc1ccccc1SSc1ccccc1Cl]

Method A: Yield=91%. ¹H NMR (400 MHz): δ=7.57 (dd, J=8.0, 1.6 Hz, 1H), 7.37 (dd, J=8.0, 1.6 Hz, 1H), 7.22 (td, J=8.0, 1.6 Hz, 1H), 7.16 (td, J=8.0, 1.6 HZ, 1H).

### Preparation of 1,2-bis(2,5-dichlorophenyl)disulfane

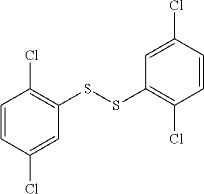 [SMILES: Clc1ccc(Cl)c(SSc2cc(Cl)ccc2Cl)c1]

Method B: Yield=72%. ¹H NMR: δ=7.52 (d, J=2.0 Hz, 1H), 7.30 (d, J=8.4 Hz, 1H), 7.15 (dd, J=8.4, 2.0 Hz, 1H).

### Preparation of 1,2-bis(2-chloro-4-fluorophenyl)disulfane

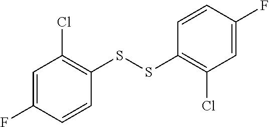 [SMILES: Fc1ccc(SSc2ccc(F)cc2Cl)c(Cl)c1]

Method A: Yield=70%. ¹H NMR: δ=7.53 (dd, J=11.6, 7.6 Hz, 1H), 7.15 (dd, J=10.8, 7.6 Hz, 1H), 6.97 (ddd, J=11.6, 10.8, 3.6 Hz, 1H).

### Preparation of 1,2-bis(2,4-dichlorophenyl)disulfane

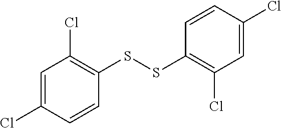 [SMILES: Clc1ccc(SSc2ccc(Cl)cc2Cl)c(Cl)c1]

Method A: Yield=92%. ¹H NMR: δ=7.46 (d, J=11.2 Hz, 1H), 7.39 (d, J=2.8 Hz, 1H), 7.21 (dd, J=11.2, 2.8 Hz, 1H).

### Preparation of 1,2-bis(2,3-dichlorophenyl)disulfane

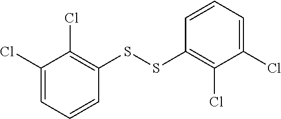 [SMILES: Clc1cccc(SSc2cccc(Cl)c2Cl)c1Cl]

Method B: Yield=73%. ¹H NMR (300 MHz): δ=7.42 (dd, J=10.8, 2.0 Hz, 1H), 7.33 (dd, J=10.8, 2.0 Hz, 1H), 7.16 (t, J=10.8 Hz, 1H).

### C. Preparation of 6-(4-morpholinophenyl)-6-(thiophen-3-yl)piperidine-2,4-dione

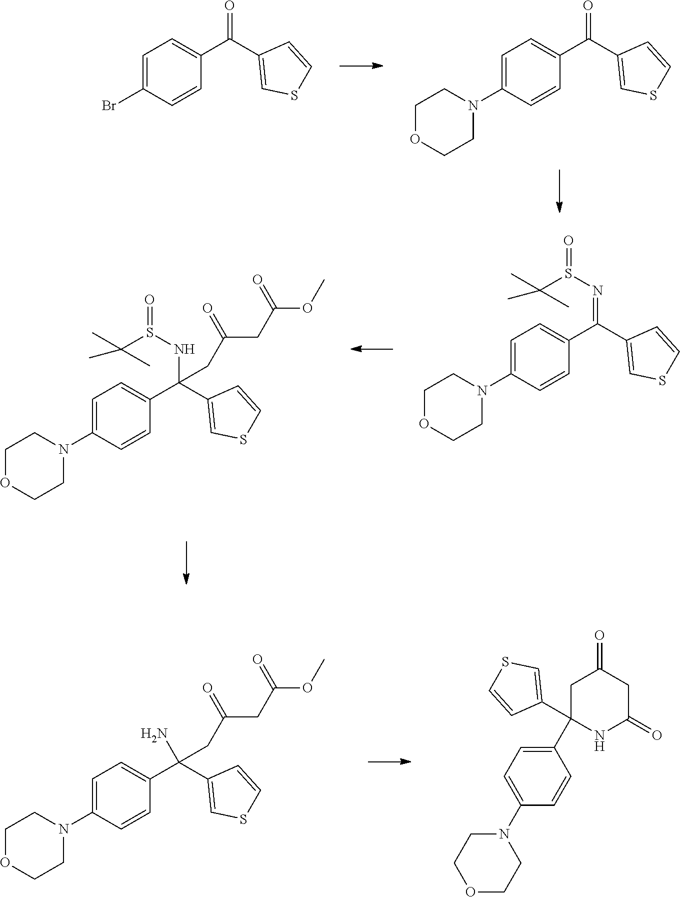 [SMILES: O=C(c1ccc(Br)cc1)c1ccsc1 ; O=C(c1ccc(N2CCOCC2)cc1)c1ccsc1 ; COC(=O)CC(=O)CC(NS(=O)C(C)(C)C)(c1ccc(N2CCOCC2)cc1)c1ccsc1 ; CC(C)(C)S(=O)/N=C(\c1ccc(N2CCOCC2)cc1)c1ccsc1 ; COC(=O)CC(=O)CC(N)(c1ccc(N2CCOCC2)cc1)c1ccsc1 ; O=C1CC(=O)NC(c2ccc(N3CCOCC3)cc2)(c2ccsc2)C1]

(4-Bromophenyl)(thiophen-3-yl)methanone was prepared according to the procedure described in WO 2015/140133.

Step A: A solution of (4-bromophenyl)(thiophen-3-yl)methanone (3.00 g, 11.2 mmol, 1 eq), morpholine (1.60 mL, 18.0 mmol, 1.5 eq), xantphos (393 mg, 0.68 mmol, 0.06 eq), Pd₂(dba)₃ (311 mg, 0.34 mmol, 0.03 eq) and K₃PO₄ (4.30 g, 20.0 mmol, 1.8 eq) in toluene (110 mL) was stirred at reflux for 18 hr. The mixture was cooled down, filtered on Celite and concentrated under reduced pressure. The crude material was purified by flash column chromatography (SiO₂, heptane/ethyl acetate: 8/2 to 2/1 to I/1) to give [4-(morpholin-4-yl)phenyl](thiophen-3-yl)methanone (2.90 g, 10.6 mmol) in 95% yield.

Step B: A solution of [4-(morpholin-4-yl)phenyl](thiophen-3-yl)methanone (5.43 g, 19.9 mmol, 1 eq), t-butylsulfinamide (7.26 g, 60.0 mmol, 3 eq) and Ti(OEt)₄ (20.9 mL, 100 mmol, 5 eq) in THF (80 mL) was stirred under reflux for 66 hr. The mixture was poured onto ice and washed with ethyl acetate (2×20 mL). The aqueous phase was extracted with ethyl acetate (2×100 mL) and the combined organic phases were dried over Na₂SO₄, filtered and concentrated under reduced pressure. The crude material was purified by flash column chromatography (SiO₂, heptane/ethyl acetate: 8/2 to 7/3 to 1/1) to give 2-methyl-N-[−[4-(morpholin-4-yl)phenyl](thiophen-3-yl)methylidene]propane-2-sulfinamide (4.74 g, 12.6 mmol) in 63% yield.

Step C: To a suspension of NaH (1.01 g, 25.2 mmol, 2 eq) in THF (50 mL) at 0° C. methyl acetoacetate (2.92 g, 25.2 mmol, 2 eq) was added. After 5 min at 0° C., n-butyllithium (10.1 mL, 25.2 mmol, 2 eq) was added and the reaction mixture was stirred for 30 min at 0° C. 2-methyl-N—[(Z)-[4-(morpholin-4-yl)phenyl](thiophen-3-yl)methylidene]propane-2-sulfinamide (4.74 g, 12.6 mmol, 1 eq) in THF (13 mL) was added and stirring continued for 1.5 hr at 0° C. TLC showed remaining starting material so another portion of reagent was prepared with methyl acetoacetate (1.3 mL, NaH (500 mg) and n-butyllithium (5.0 mL) and added to the reaction mixture. After 1.5 h at 0° C., the reaction was stopped by the addition of saturated aqueous NH₄Cl (20 mL). The phases were separated and the aqueous phase was extracted with ethyl acetate (2×50 mL) and the combined organic phases were washed with brine (40 mL), saturated aqueous NaHCO₃ (40 mL) and HCl 1M (40 mL), dried over Na₂SO₄, filtered and concentrated under reduced pressure. The crude material was purified by flash column chromatography (SiO₂, heptane/ethyl acetate: 3/1 to 2/1 to 1/1 to 1/3 to ethyl acetate) to give methyl 5-((tert-butylsulfinyl)amino)-5-(4-morpholinophenyl)-3-oxo-5-(thiophen-3-yl)pentanoate (3.40 g, 6.90 mmol) in 55% yield.

Step D: To a solution of methyl 5-((tert-butylsulfinyl)amino)-5-(4-morpholinophenyl)-3-oxo-5-(thiophen-3-yl)pentanoate (3.40 g, 6.90 mmol, 1 eq) in methanol (69 mL) TMSCl (2.62 mL, 20.7 mmol, 3 eq) was added. The reaction mixture was stirred for 1 hr at room temperature. The reaction was stopped by the addition of aqueous NaOH 2M (11 mL) and the methanol was removed under reduced pressure. The aqueous phase was extracted with ethyl acetate (3×50 mL) and the combined organic phases were dried over Na₂SO₄, filtered and concentrated under reduced pressure to give the crude product (2.65 g), which was used directly into the next step.

Step E: A solution of methyl 5-amino-5-(4-morpholinophenyl)-3-oxo-5-(thiophen-3-yl)pentanoate (2.65 g, 6.82 mmol, 1 eq) and K₂CO₃ (2.83 g, 20.5 mmol, 3 eq) in methanol (34 mL) was stirred at reflux for 2 hr. The mixture was concentrated under reduced pressure and diluted in aqueous HCl 1M (30 mL). The aqueous phase was extracted with ethyl acetate (3×50 mL) and the combined organic phases were dried over Na₂SO₄, filtered and concentrated under reduced pressure. The crude material was purified by flash column chromatography (SiO₂, heptane/ethyl acetate: 4/1 to 2/1 to 1/1 to 1/3 to ethyl acetate to 2% MeOH) to give 6-(4-morpholinophenyl)-6-(thiophen-3-yl)piperidine-2,4-dione (726 mg, 1.87 mmol) in 30% yield over 2 steps.

### D. Preparation of 6-(6-bromopyridin-2-yl)-3-((2-chlorophenyl)thio)-6-(thiophen-3-yl)piperidine-2,4-dione (Example 1-8 in WO 2015/142903)

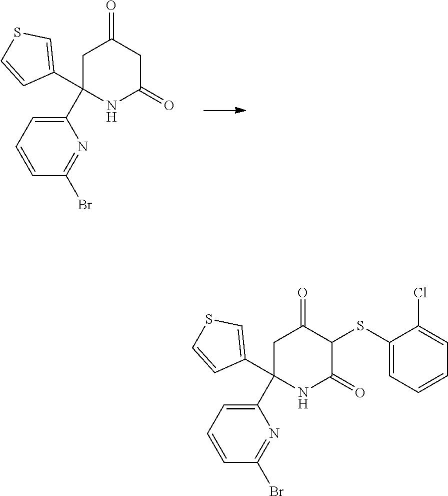 [SMILES: O=C1CC(=O)NC(c2ccsc2)(c2cccc(Br)n2)C1 ; O=C1CC(c2ccsc2)(c2cccc(Br)n2)NC(=O)C1Sc1ccccc1Cl]

6-(6-Bromopyridin-2-yl)-3-((2-chlorophenyl)thio)-6-(thiophen-3-yl)piperidine-2,4-dione was prepared according to the procedure described in WO 2015/142903.

Potassium carbonate (7.5 g, 54 mmol) and 1,2-bis(2-chlorophenyl)disulfane (3.1 g, 10.8 mmol) were added to a stirred solution of 6-(6-bromopyridin-2-yl)-6-(thiophen-3-yl)piperidine-2,4-dione (6.3 g, 17.9 mmol) in methanol (150 mL). The mixture was heated a reflux under an argon atmosphere for 2 hr, after which it was allowed to cool to room temperature. The methanol was evaporated and the resulting mixture was partitioned between water (100 mL) and ethyl acetate (30 mL). The layers were separated and the aqueous was extracted with ethyl acetate (3×30 mL). The organic layers were discarded and the aqueous was acidified to pH 4 by the addition of 3M hydrochloric acid (40 mL). This was then extracted with ethyl acetate (4×40 mL). These organic fractions were combined, washed with brine and dried over sodium sulfate. It was then filtered and the solvent was removed under reduced pressure.

The crude mixture was then subjected to column chromatography (hexane/ethyl acetate, 1/2), which provided the product (4.6 g) as a pale tan colored solid. The ¹H NMR spectrum was consistent with that reported.

### Preparation of Final Compounds

### Example 1—Preparation of 6-[6-(cyclopentylmethoxy)pyridin-2-yl]-3-[(2,4-dichlorophenyl)sulfanyl]-6-(thiophen-3-yl)piperidine-2,4-dione

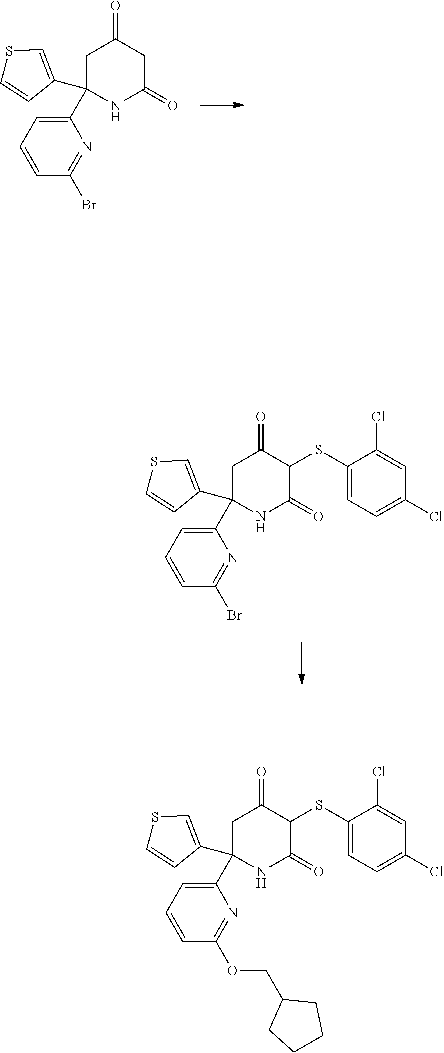 [SMILES: O=C1CC(=O)NC(c2ccsc2)(c2cccc(Br)n2)C1 ; O=C1CC(c2ccsc2)(c2cccc(Br)n2)NC(=O)C1Sc1ccc(Cl)cc1Cl ; O=C1CC(c2ccsc2)(c2cccc(OCC3CCCC3)n2)NC(=O)C1Sc1ccc(Cl)cc1Cl]

Step A: To a solution of 6-(6-bromopyridin-2-yl)-6-(thiophen-3-yl)piperidine-2,4-dione (500 mg, 1.4 mmol, 1 eq) in MeOH (14 mL) 1,2-bis(2,4-dichlorophenyl) disulfane (303 mg, 0.85 mmol, 0.6 eq) and potassium carbonate (593 mg, 4.3 mmol, 3 eq) were added. The reaction was stirred for 2 hr under reflux, and concentrated under reduced pressure. Water (10 mL) and HCl 1M (7 mL) were added and the aqueous phase was extracted with ethyl acetate (3×15 mL). The combined organic phases were dried with Na₂SO₄, filtered and concentrated under reduced pressure. The crude product was purified by flash chromatography on silica gel (eluent: heptane/ethyl acetate: 70/30 to 30/70) to give 6-(6-bromopyridin-2-yl)-3-[(2,4-dichlorophenyl)sulfanyl]-6-(thiophen-3-yl)piperidine-2,4-dione (487 mg, 0.92 mmol) in 65% yield.

Step B: To a suspension of NaH (76 mg, 1.9 mmol, 5 eq) in THF (4 mL) at 0° C. cyclopentanemethanol (204 μL, 0.9 mmol, 5 eq) was added. The reaction was stirred at 0° C. for 30 min and 6-(6-bromopyridin-2-yl)-3-[(2,4-dichlorophenyl)sulfanyl]-6-(thiophen-3-yl)piperidine-2,4-dione (200 mg, 0.38 mmol, 1 eq) was added. The reaction was stirred overnight under reflux and quenched by the addition of water (10 mL) and HCl 1M (5 mL). The aqueous phase was extracted with ethyl acetate (3×15 mL) and the combined organic phases were dried with Na₂SO₄, filtered and concentrated under reduced pressure. The crude product was purified by flash chromatography on silica gel (eluent: heptane/ethyl acetate: 75/25) to give 6-[6-(cyclopentylmethoxy)pyridin-2-yl]-3-[(2,4-dichlorophenyl)sulfanyl]-6-(thiophen-3-yl)piperidine-2,4-dione (115 mg, 0.21 mmol) in 55% yield.

¹H NMR (400 MHz, MeOD): δ=7.75 (t, J=8.0 Hz, 1H), 7.49 (dd, J=9.2, 3.2 Hz, 1H), 7.35 (d, J=2.0 Hz, 1H), 7.32 (dd, J=6.8, 1.2 Hz), 7.19-7.17 (m, 2H), 6.80-6.76 (m, 2H), 5.94 (d, J=8.0 Hz, 1H), 4.29-4.21 (m, 2H), 3.91 (d, J=16.4 Hz, 1H), 3.47 (d, J=16.4 Hz, 11-1), 2.35-2.29 (m, 1H), 1.85-1.77 (m, 2H), 1.68-1.54 (m, 4H), 1.41-1.31 (m, 21H).

### Example 2—Preparation of 3-[(2-chloro-4-fluorophenyl)sulfanyl]-6-[6-(cyclopentylmethoxy)pyridin-2-yl]-6-(thiophen-3-yl)piperidine-2,4-dione

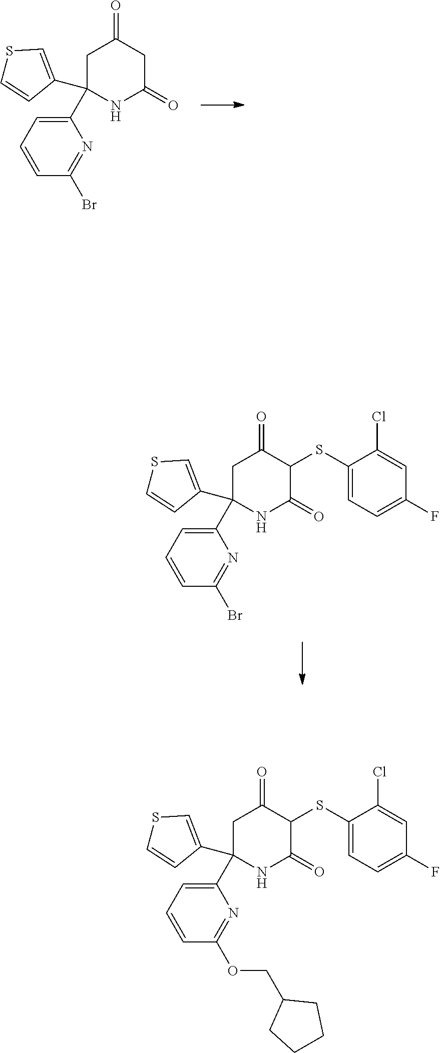 [SMILES: O=C1CC(=O)NC(c2ccsc2)(c2cccc(Br)n2)C1 ; O=C1CC(c2ccsc2)(c2cccc(Br)n2)NC(=O)C1Sc1ccc(F)cc1Cl ; O=C1CC(c2ccsc2)(c2cccc(OCC3CCCC3)n2)NC(=O)C1Sc1ccc(F)cc1Cl]

Step A: To a solution of 6-(6-bromopyridin-2-yl)-6-(thiophen-3-yl)piperidine-2,4-dione (500 mg, 1.4 mmol, 1 eq) in MeOH (14 mL) 1,2-bis(2-chloro-4-fluorophenyl)disulfane (550 mg, 1.7 mmol, 1.2 eq) and potassium carbonate (593 mg, 4.3 mmol, 3 eq) were added. The reaction was stirred for 2 hr under reflux, and concentrated under reduced pressure. Water (10 mL) and HCl 1M (7 mL) were added and the aqueous phase was extracted with ethyl acetate (3×15 mL). The combined organic phases were dried with Na₂SO₄, filtered and concentrated under reduced pressure. The crude product was purified by flash chromatography on silica gel (eluent: heptane/ethyl acetate: 70/30 to 30/70) to give 6-(6-bromopyridin-2-yl)-3-[(2-chloro-4-fluorophenyl)sulfanyl]-6-(thiophen-3-yl)piperidine-2,4-dione (466 mg, 0.91 mmol) in 64% yield.

¹H NMR (DMSO-d6, 400 MHz): δ=11.79 (br s, 1H), 8.58 (s, 1H), 7.88 (t, J=8.0 Hz, 1H), 7.68 (dd, J=8.8, 8.0 Hz, 2H), 7.56 (dd, J=5.2, 3.2 Hz, 1H), 7.36 (dd, J=8.8, 2.8 Hz, 1H), 7.34 (dd, J=2.8, 1.2 Hz, 1H), 7.15 (dd, J=8.8, 1.2 Hz, 1H), 6.72 (td, J=8.8, 2.8 Hz, 1H), 5.95 (dd, J=8.8, 6.0 Hz, 1H), 3.82 (d, J=16.4 Hz, 1H), 3.42 (d, J=16.4 Hz, 1H).

¹³C NMR (DMSO-d6, 100 MHz): δ=166.1, 164.4, 158.9 (d, J=242 Hz), 144.4, 140.7, 140.1, 133.2 (d, J=4 Hz), 129.3 (d, J=10 Hz), 127.0, 127.0, 126.4, 125.8 (d, J=7.7 Hz), 122.2, 120.6, 116.5 (d, J=25 Hz), 114.2 (J=21 Hz), 60.5.

Step B: To a suspension of NaH (98 mg, 2.5 mmol, 5 eq) in THF (5 mL) at 0° C. cyclopentanemethanol (263 μL, 2.5 mmol, 5 eq) was added. The reaction was stirred at 0° C. for 30 min and 6-(6-bromopyridin-2-yl)-3-[(2-chloro-4-fluorophenyl)sulfanyl]-6-(thiophen-3-yl)piperidine-2,4-dione (250 mg, 0.49 mmol, 1 eq) was added. The reaction was then stirred overnight under reflux and quenched by the addition of water (10 mL) and HCl 1M (5 mL). The aqueous phase was extracted with ethyl acetate (3×15 mL) and the combined organic phases were dried with Na₂SO₄, filtered and concentrated under reduced pressure. The crude product was purified by flash chromatography on silica gel (eluent: heptane/ethyl acetate: 75/25) to give 3-[(2-chloro-4-fluorophenyl)sulfanyl]-6-[6-(cyclopentylmethoxy)pyridin-2-yl]-6-(thiophen-3-yl)piperidine-2,4-dione (192 mg, 0.36 mmol) in 74% yield.

¹H NMR (MeOD-d4, 400 MHz): δ=7.71 (dd, J=8.0, 7.6 Hz, 1H), 7.44 (dd, J=8.8, 7.2 Hz, 1H), 7.27 (dd, J=2.8, 1.2 Hz, 1H), 7.15-7.11 (m, 2H), 7.09 (dd, J=4.8, 2.8 Hz, 1H), 6.75 (d, J=8.4 Hz, 1H), 6.54 (td, J=8.4, 2.8 Hz, 1H), 5.99 (dd, J=8.8, 6.0 Hz, 1H), 4.27-4.18 (m, 2H), 3.87 (d, J=16.4 Hz, 1H), 3.45 (d, J=16.4 Hz, 1H), 2.36-2.28 (m, 1H), 1.83-1.74 (m, 2H), 1.66-1.53 (m, 4H), 1.39-1.29 (m, 2H).

¹³C NMR (MeOD-d4, 100 MHz): δ=166.9, 161.6, 158.6 (d, J=245 Hz), 157.2, 143.5, 138.2, 130.7 (d, J=4 Hz), 124.8 (d, J=8.5 Hz), 124.7, 124.5, 120.1, 114.7 (d, J=25 Hz), 112.3 (d, J=21 Hz), 111.8, 108.0, 100.0, 68.3, 59.3, 39.1, 37.3, 27.5, 23.4.

### Example 3—Preparation of 6-[6-(cyclopentylmethoxy)pyridin-2-yl]-3-[(2,5-dichlorophenyl)sulfanyl]-6-(thiophen-3-yl)piperidine-2,4-dione

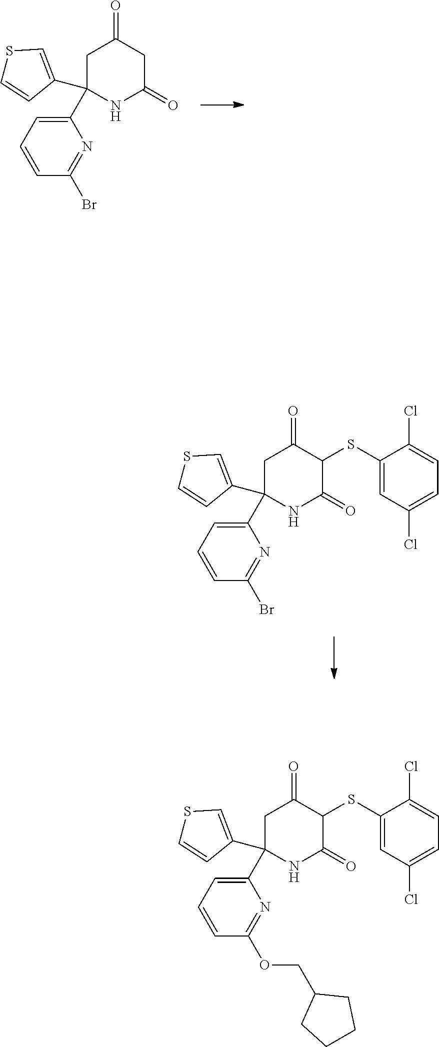 [SMILES: O=C1CC(=O)NC(c2ccsc2)(c2cccc(Br)n2)C1 ; O=C1CC(c2ccsc2)(c2cccc(Br)n2)NC(=O)C1Sc1cc(Cl)ccc1Cl ; O=C1CC(c2ccsc2)(c2cccc(OCC3CCCC3)n2)NC(=O)C1Sc1cc(Cl)ccc1Cl]

Step A: To a solution of 6-(6-bromopyridin-2-yl)-6-(thiophen-3-yl)piperidine-2,4-dione (790 mg, 2.3 mmol, 1 eq) in MeOH (23 mL) 1,2-bis(2,5-dichlorophenyl) disulfane (480 mg, 1.4 mmol, 0.6 eq) and potassium carbonate (930 mg, 6.8 mmol, 3 eq) were added. The reaction was stirred for 2 hr under reflux, then concentrated under reduced pressure. Water (10 mL) and HCl 1M (7 mL) were added and the aqueous phase was extracted with ethyl acetate (3×15 mL). The combined organic phases were dried with Na₂SO₄, filtered and concentrated under reduced pressure. The crude product was purified by flash chromatography on silica gel (eluent: heptane/ethyl acetate: 70/30 to 30/70) to give 6-(6-bromopyridin-2-yl)-3-[(2,5-dichlorophenyl)sulfanyl]-6-(thiophen-3-yl)piperidine-2,4-dione (179 mg, 0.34 mmol) in 15% yield.

¹H NMR (MeOD-d4, 400 MHz): δ=7.75 (t, J=7.6 Hz, 1H), 7.61-7.56 (m, 2H), 7.45 (br s, 1H), 7.24-7.22 (m, 2H), 7.11 (d, J=1.2 Hz, 1H), 6.97 (d, J=8.4 Hz, 1H), 6.24 (s, 1H), 3.98 (d, J=16.4 Hz, 1H), 3.46 (d, J=16.4 Hz, 1H).

Step B: To a suspension of NaH (64 mg, 1.6 mmol, 5 eq) in THF (3.5 mL) at 0° C. cyclopentanemethanol (172 μL, 1.6 mmol, 5 eq) was added. The reaction was stirred at 0° C. for 30 min and 6-(6-bromopyridin-2-yl)-3-[(2,5-dichlorophenyl)sulfanyl]-6-(thiophen-3-yl)piperidine-2,4-dione (170 mg, 0.32 mmol, 1 eq) was added. The reaction was then stirred overnight under reflux and quenched by the addition of water (10 mL) and HCl 1M (5 mL). The aqueous phase was extracted with ethyl acetate (3×15 mL) and the combined organic phases were dried with Na₂SO₄, filtered and concentrated under reduced pressure. The crude product was purified by flash chromatography on silica gel (eluent: heptane/ethyl acetate: 75/25) to give 6-[6-(cyclopentylmethoxy)pyridin-2-yl]-3-[(2,5-dichlorophenyl)sulfanyl]-6-(thiophen-3-yl)piperidine-2,4-dione (104 mg, 0.19 mmol) in 59% yield.

¹H NMR (MeOD-d4, 400 MHz): δ=7.69 (t, J=8.0 Hz, 1H), 7.41 (dd, J=4.8, 3.2 Hz, 1-1), 7.24-7.21 (m, 2H), 7.12-7.09 (m, 2H), 6.96 (dd, J=8.8, 2.8 Hz, 1H), 6.71 (d, J=8.0 Hz, 1H), 6.26 (d, J=2.4 Hz, 1H), 4.28-4.18 (m, 2H), 9.25 (d, J=16.4 Hz, 1H), 3.42 (d, J=16.4 Hz, 1H), 2.36-2.28 (m, 1H), 1.83-1.74 (m, 2H), 1.65-1.52 (m, 4H), 1.40-1.29 (m, 2H).

¹³C NMR (MeOD-d4, 100 MHz): δ=166.5, 161.6, 158.0, 143.7, 138.3, 137.5, 131.1, 128.5, 127.0, 124.7, 124.3, 123.5, 123.0, 119.9, 111.5, 68.2, 59.1, 39.1, 37.4, 27.6, 23.5.

### Example 4—Preparation of 6-[6-(cyclopentylmethoxy)pyridin-2-yl]-3-[(2,3-dichlorophenyl)sulfanyl]-6-(thiophen-3-yl)piperidine-2,4-dione

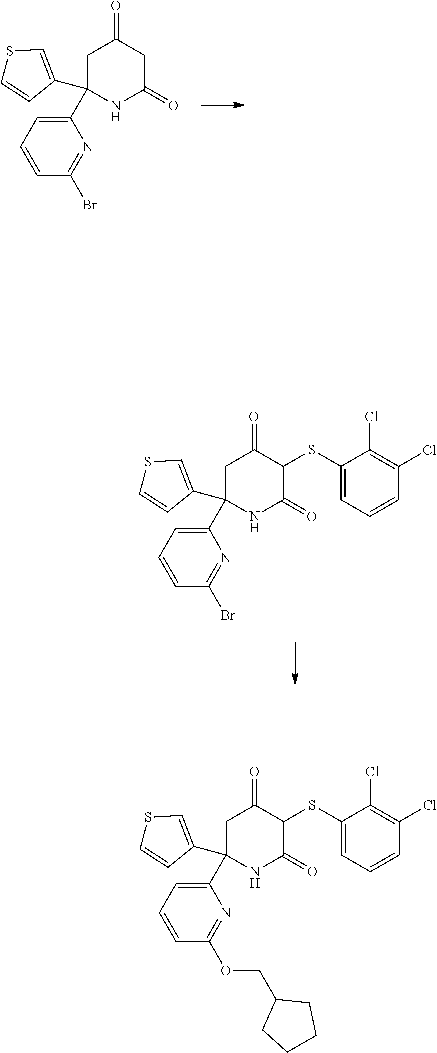 [SMILES: O=C1CC(=O)NC(c2ccsc2)(c2cccc(Br)n2)C1 ; O=C1CC(c2ccsc2)(c2cccc(Br)n2)NC(=O)C1Sc1cccc(Cl)c1Cl ; O=C1CC(c2ccsc2)(c2cccc(OCC3CCCC3)n2)NC(=O)C1Sc1cccc(Cl)c1Cl]

Step A: To a solution of 6-(6-bromopyridin-2-yl)-6-(thiophen-3-yl)piperidine-2,4-dione (400 mg, 1.1 mmol, 1 eq) in MeOH (11 mL) 1,2-bis(2,3-dichlorophenyl) disulfane (463 mg, 1.3 mmol, 1.2 eq) and potassium carbonate (456 mg, 3.3 mmol, 3 eq) were added. The reaction was stirred for 2 hr under reflux, and concentrated under reduced pressure. Water (10 mL) and HCl 1M (7 mL) were added and the aqueous phase was extracted with ethyl acetate (3×15 mL). The combined organic phases were dried with Na₂SO₄, filtered and concentrated under reduced pressure. The crude product was purified by flash chromatography on silica gel (eluent: heptane/ethyl acetate: 70/30 to 30/70) to give 6-(6-bromopyridin-2-yl)-3-[(2,3-dichlorophenyl)sulfanyl]-6-(thiophen-3-yl)piperidine-2,4-dione (447 mg, 0.85 mmol) in 77% yield.

¹H NMR (MeOD-d4, 400 MHz): δ=7.74 (t, J=8.0 Hz, 1H), 7.59 (t, J=7.6 Hz, 1H), 7.47 (dd, J=5.2, 3.2 Hz, 1H), 7.30 (dd, J=2.8, 1.6 Hz, 1H), 7.15 (d, J=1.2 Hz, 1H), 7.14 (dd, J=2.8, 1.2 Hz, 1H), 6.79 (t, J=8.0 Hz, 1H), 5.99 (d, J=8.0, 1H), 3.89 (d, J=16.4 Hz, 1H), 3.49 (d, J=16.4 Hz, 1H).

¹³C NMR (MeOD-d4, 100 MHz): δ=176.6, 169.4, 164.9, 145.7, 142.1, 141.3, 141.0, 134.0, 129.2, 128.6, 128.4, 128.0, 127.3, 127.0, 124.6, 123.5, 121.6, 95.5, 62.1, 41.9, 14.5.

Step B: To a suspension of NaH (56 mg, 1.4 mmol, 5 eq) in THF (3.5 mL) at 0° C. cyclopentanemethanol (151 μL, 1.4 mmol, 5 eq) was added. The reaction was stirred at 0° C. for 30 min and 6-(6-bromopyridin-2-yl)-3-[(2,3-dichlorophenyl)sulfanyl]-6-(thiophen-3-yl)piperidine-2,4-dione (150 mg, 0.28 mmol, 1 eq) was added. The reaction was then stirred overnight under reflux and quenched by the addition of water (10 mL) and HCl 1M (5 mL). The aqueous phase was extracted with ethyl acetate (3×15 mL) and the combined organic phases were dried with Na₂SO₄, filtered and concentrated under reduced pressure. The crude product was purified by flash chromatography on silica gel (eluent: heptane/ethyl acetate: 75/25 to 50/50) to give 6-[6-(cyclopentylmethoxy)pyridin-2-yl]-3-[(2,3-dichlorophenyl)sulfanyl]-6-(thiophen-3-yl)piperidine-2,4-dione (73 mg, 0.13 mmol) in 48% yield.

¹H NMR (MeOD-d4, 400 MHz): δ=7.71 (t, J=8.0 Hz, 1H), 7.45-7.43 (m, 1H), 7.28-7.27 (m, 1H), 7.15-7.11 (m, 3H), 6.76-6.69 (m, 2H), 5.90 (d, J=8.0 Hz, 1H), 4.27-4.18 (m, 1H), 3.88 (d, J=16.4 Hz, 1H), 3.45 (d, J=16.4 Hz, 1H), 2.35-2.27 (m, 2H), 1.80-1.74 (m, 2H), 1.63-1.52 (m, 4H), 1.37-1.30 (m, 2H).

¹³C NMR (MeOD-d4, 100 MHz): δ=169.8, 164.5, 161.1, 146.4, 141.2, 133.9, 129.1, 128.4, 127.6, 127.4, 126.9, 124.6, 123.0, 114.8, 110.9, 71.2, 62.2, 42.1, 40.2, 30.5, 26.3.

### Example 5—Preparation of 3-((2-chloro-4-fluorophenyl)thio)-6-(4-morpholinophenyl)-6-(thiophen-3-yl)piperidine-2,4-dione

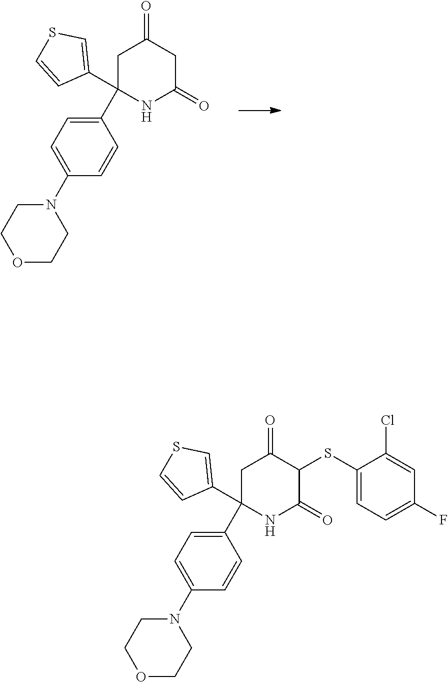 [SMILES: O=C1CC(=O)NC(c2ccc(N3CCOCC3)cc2)(c2ccsc2)C1 ; O=C1CC(c2ccc(N3CCOCC3)cc2)(c2ccsc2)NC(=O)C1Sc1ccc(F)cc1Cl]

A solution of 6-(4-morpholinophenyl)-6-(thiophen-3-yl)piperidine-2,4-dione (60 mg, 0.17 mmol), 1,2-bis(2-chloro-4-fluorophenyl)disulfane (65 mg, 0.20 mmol) and K₂CO₃ (70 mg, 0.50 mmol) in methanol (2 mL) was stirred at reflux for 2 hr. The mixture was concentrated under reduced pressure and diluted in water (3 mL) and aqueous HCl 1M (2 mL). The aqueous phase was extracted with ethyl acetate (3×10 mL) and the combined organic phases dried over Na₂SO₄, filtered and concentrated under reduced pressure. The crude material was purified by flash column chromatography (SiO₂, heptane/ethyl acetate: 20 to 40 to 60 to 70%) to give 3-((2-chloro-4-fluorophenyl)thio)-6-(4-morpholinophenyl)-6-(thiophen-3-yl)piperidine-2,4-dione in 87% yield.

¹H NMR (400 MHz, MeOD): δ=7.56 (dd, J=5.2, 2.8 Hz, 1H), 7.35 (d, J=8.8 Hz, 2H), 7.32-7.31 (m, 1H), 7.21-7.16 (m, 2H), 7.02 (d, J=8.8 Hz, 2H), 7.60 (td, J=8.4, 2.4 Hz, 1H), 5.96 (dd, J=8.8, 5.6 Hz, 1H), 3.85 (dd, J=4.8, 4.8 Hz, 4H), 3.28 (s, 2H), 3.20 (dd, J=5.2, 4.4 Hz, 4H).

### Example 6—Preparation of 3-((2,5-dichlorophenyl)thio)-6-(4-morpholinophenyl)-6-(thiophen-3-yl)piperidine-2,4-dione

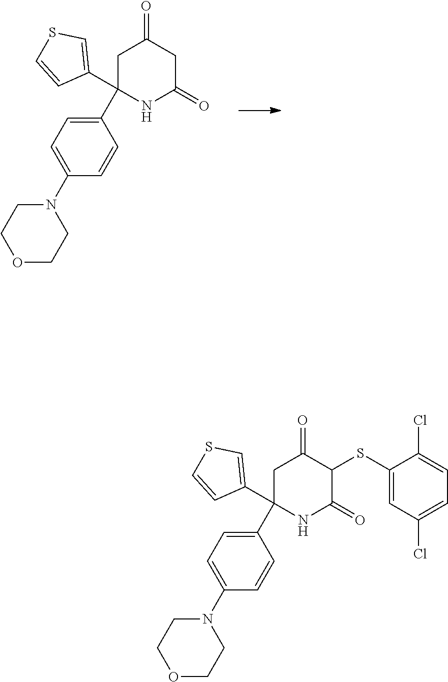 [SMILES: O=C1CC(=O)NC(c2ccc(N3CCOCC3)cc2)(c2ccsc2)C1 ; O=C1CC(c2ccc(N3CCOCC3)cc2)(c2ccsc2)NC(=O)C1Sc1cc(Cl)ccc1Cl]

This compound was prepared according to Example 5, using 6-(4-morpholinophenyl)-6-(thiophen-3-yl)piperidine-2,4-dione and 1,2-bis(2,5-dichlorophenyl)disulfane, in 55% yield.

¹H NMR (400 MHz, MeOD): δ=7.50 (dd, J=5.2, 3.2 Hz, 1H), 7.33 (d, J=8.8 Hz, 2H), 7.28 (d, J=8.4 Hz, 1H), 7.26 (dd, J=2.8, 1.2 Hz, 1H), 7.14 (d, J=4.8, 1.2 Hz, 1H), 7.03-7.01 (m, 1H), 7.00 (d, J=9.2 Hz, 2H), 6.30 (d, J=2.8 Hz), 3.84-3.82 (m, 4H), 3.50 (s, 2H), 3.20-3.17 (m, 4H).

The compound of Example 6 was tested in the assays as the racemate, and additionally as single enantiomers. It was possible to acquire the individual enantiomers by chiral preparative HPLC of the final product using an ethanol/acetonitrile/diethylamine (90/10/0.1) solvent system. Analysis of the enantiomers by analytical HPLC on a ChiralPak IC column using the same solvent system revealed that the enantiomers had been isolated in 100.0% (Enantiomer 1: Rt=5.0 min) and 99.4% (Enantiomer 2: Rt=7.0 min) e.e.

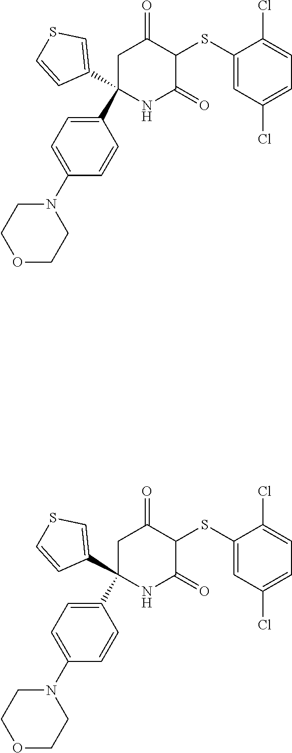 [SMILES: O=C1C[C@@](c2ccc(N3CCOCC3)cc2)(c2ccsc2)NC(=O)C1Sc1cc(Cl)ccc1Cl ; O=C1C[C@](c2ccc(N3CCOCC3)cc2)(c2ccsc2)NC(=O)C1Sc1cc(Cl)ccc1Cl]

### Example 7—Preparation of 3-((2-chlorophenyl)thio)-6-(4-morpholinophenyl)-6-(thiophen-3-yl)piperidine-2,4-dione (Example 44 in WO 2015/142903)

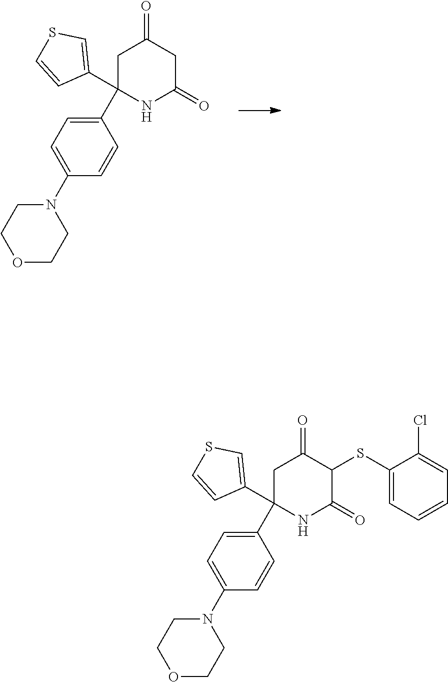 [SMILES: O=C1CC(=O)NC(c2ccc(N3CCOCC3)cc2)(c2ccsc2)C1 ; O=C1CC(c2ccc(N3CCOCC3)cc2)(c2ccsc2)NC(=O)C1Sc1ccccc1Cl]

A solution of 6-(4-morpholinophenyl)-6-(thiophen-3-yl)piperidine-2,4-dione (50 mg, 0.14 mmol), 1,2-bis(2-chlorophenyl)disulfane (48 mg, 0.17 mmol) and K₂CO₃ (58 mg, 0.42 mmol) in methanol (1.5 mL) was stirred at reflux for 2 hr. The mixture was concentrated under reduced pressure and diluted in water (3 mL) and aqueous HCl 1M (1 mL). The aqueous phase was extracted with ethyl acetate (3×5 mL) and the combined organic phases were dried over Na₂SO₄, filtered and concentrated under reduced pressure. The crude material was purified by flash column chromatography (SiO₂, heptane/ethyl acetate: 2/1 to 1/1 to 1/3) to give 3-((2-chlorophenyl)thio)-6-(4-morpholinophenyl)-6-(thiophen-3-yl)piperidine-2,4-dione in 61% yield. Analytical data were identical to the literature (ACS Med. Chem. Lett. 7: 896-901, 2016).

The compound of Example 7 was tested in the assays as the racemate, and additionally as single enantiomers. It was possible to acquire the individual enantiomers by chiral preparative HPLC of the final product using an ethanol/acetonitrile/diethylamine (90/10/0.1) solvent system. Analysis of the enantiomers by analytical HPLC on a ChiralPak IC column using the same solvent system revealed that the enantiomers had been isolated in 100% (Enantiomer 1: Rt=5.5 min) and 97% (Enantiomer 2: Rt=7.5 min) e.e.

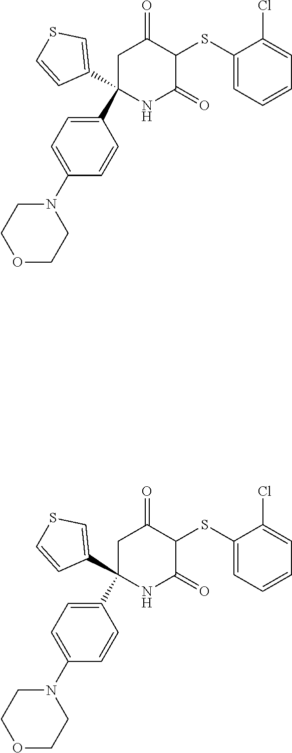 [SMILES: O=C1C[C@@](c2ccc(N3CCOCC3)cc2)(c2ccsc2)NC(=O)C1Sc1ccccc1Cl ; O=C1C[C@](c2ccc(N3CCOCC3)cc2)(c2ccsc2)NC(=O)C1Sc1ccccc1Cl]

### Example 8—Preparation of 3-((2-chlorophenyl)thio)-6-(6-(cyclopentylmethoxy)pyridin-2-yl)-6-(thiophen-3-yl)piperidine-2,4-dione (Example 194 in WO 2015/142903)

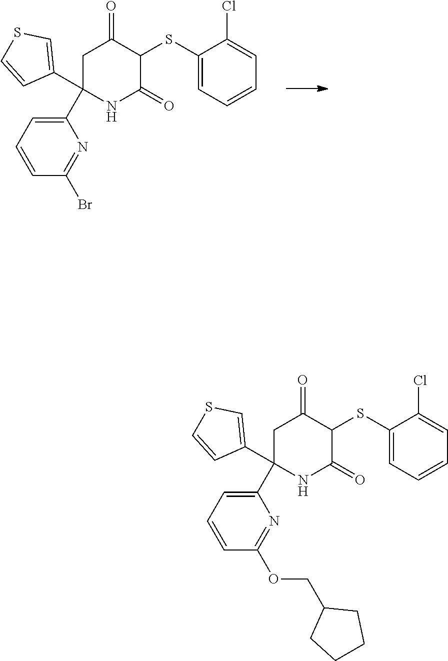 [SMILES: O=C1CC(c2ccsc2)(c2cccc(Br)n2)NC(=O)C1Sc1ccccc1Cl ; O=C1CC(c2ccsc2)(c2cccc(OCC3CCCC3)n2)NC(=O)C1Sc1ccccc1Cl]

To a suspension of NaH (61 mg, 1.5 mmol) in THF (3 mL) at 0° C. cyclopentanemethanol (0.16 mL, 1.5 mmol) was added. After 30 min at 0° C., 6-(6-bromopyridin-2-yl)-3-((2-chlorophenyl)thio)-6-(thiophen-3-yl)piperidine-2,4-dione (150 mg, 0.30 mmol) was added and the reaction mixture was stirred for 18 hr at reflux. The reaction was stopped by the addition of water (10 mL) and HCl 1M (3 mL). The aqueous phase was extracted with ethyl acetate (3×10 mL) and the combined organic phases were dried over Na₂SO₄, filtered and concentrated under reduced pressure. The crude material was purified by flash column chromatography (SiO₂, heptane/ethyl acetate: 8/2 to 7/3 to 1/1) to give 3-((2-chlorophenyl)thio)-6-(6-(cyclopentylmethoxy)pyridin-2-yl)-6-(thiophen-3-yl)piperidine-2,4-dione in 62% yield.

¹H NMR (400 MHz, MeOH-d4): δ=7.70 (t, J=7.8 Hz, 1H), 7.43 (dd, J=5.0, 3.0 Hz, 1H), 7.28 (br s, 1H), 7.22 (d, J=7.9 HZ, 1H), 7.15-7.12 (m, 2H), 6.94 (t, J=7.8 Hz, 1H), 6.77-6.73 (m, 2H), 5.98 (d, J=8.0 Hz, 1H), 4.22 (m, 2H9, 3.91 (d, J=16.4 Hz, 1H), 3.45 (d, J=16.4 Hz, 1H), 3.45 (s, 1H), 2.35-2.28 (m, 1H), 1.82-1.73 (m, 2H), 1.64-1.51 (m, 4H), 1.38-1.30 (m, 2H).

### Example 9—Preparation of 5-((2,5-dichlorophenyl)thio)-2-(4-morpholinophenyl)-6-oxo-2-(thiophen-3-yl)-1,2,3,6-tetrahydropyridin-4-yl isonicotinate

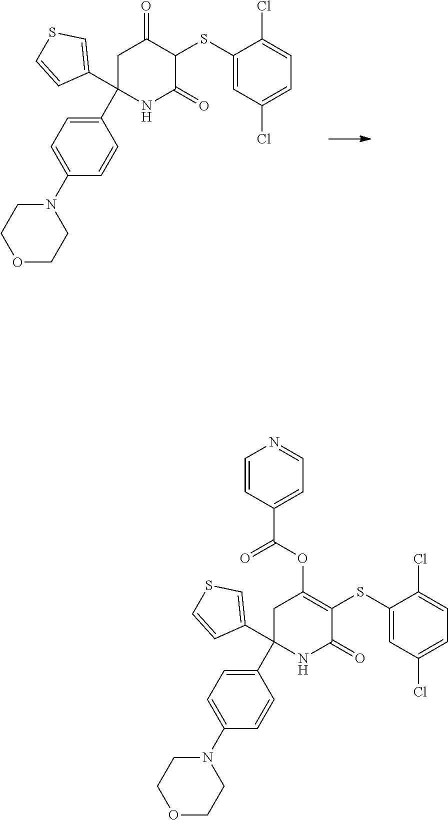 [SMILES: O=C1CC(c2ccc(N3CCOCC3)cc2)(c2ccsc2)NC(=O)C1Sc1cc(Cl)ccc1Cl ; O=C1NC(c2ccc(N3CCOCC3)cc2)(c2ccsc2)CC(OC(=O)c2ccncc2)=C1Sc1cc(Cl)ccc1Cl]

To a solution of 3-((2,5-dichlorophenyl)thio)-6-(4-morpholinophenyl)-6-(thiophen-3-yl)piperidine-2,4-dione (0.15 mmol) in DCM (1.5 mL) at 0° C. was added diisopropylethylamine (0.23 mmol). After 5 min at 0° C., isonicotinoyl chloride hydrochloride (0.18 mmol) was added and the reaction was stirred at 0° C. for 1.5 hr. The reaction was quenched by the addition of a saturated aqueous solution of NH₄Cl (2 mL) and water (5 mL) and the aqueous phase was extracted with ethyl acetate (3×10 mL). The combined organic phases were dried with Na₂SO₄, filtered and concentrated in vacuo. The crude product was purified by flash chromatography on silica gel (eluent: heptane/ethyl acetate: 80/20 to 50/50) to give 5-((2,5-dichlorophenyl)thio)-2-(4-morpholinophenyl)-6-oxo-2-(thiophen-3-yl)-1,2,3,6-tetrahydropyridin-4-yl isonicotinate in 42% yield.

¹H NMR (300 MHz, CDCl₃): δ=8.79 (br s, 2H), 7.73 (d, J=5.0 Hz, 2H), 7.41-7.38 (m, 1H), 7.29 (br s, 1H), 7.22 (br s, 1H), 7.10 (d, J=8.5 Hz, 1H), 7.01-6.94 (m, 4H), 6.64 (d, J=2.3 Hz, 2H), 3.90-3.87 (m, 4H), 3.67 (s, 2H), 3.22-3.19 (m, 4H).

### Example 10—Preparation of 3-((2-chlorophenyl)thio)-6-(6-(oxetan-3-yloxy)pyridin-2-yl)-6-(thiophen-3-yl)piperidine-2,4-dione

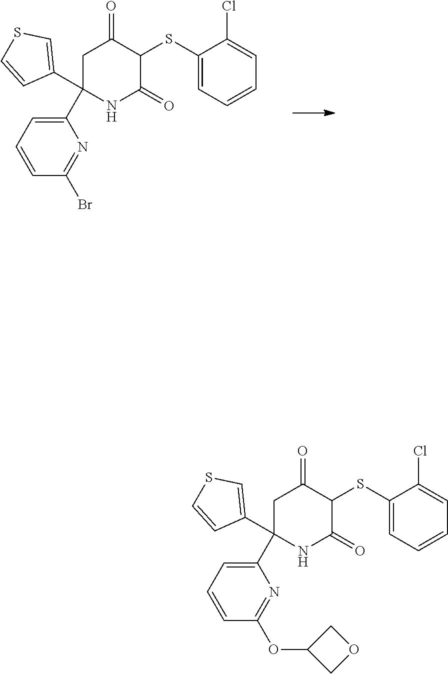 [SMILES: O=C1CC(c2ccsc2)(c2cccc(Br)n2)NC(=O)C1Sc1ccccc1Cl ; O=C1CC(c2ccsc2)(c2cccc(OC3COC3)n2)NC(=O)C1Sc1ccccc1Cl]

To a suspension of NaH (49 mg, 1.22 mmol) in THF (2.4 mL) at 0° C. was added oxetan-3-ol (80 μL, 1.22 mmol). After 30 min at 0° C., 6-(6-bromopyridin-2-yl)-3-((2-chlorophenyl)thio)-6-(thiophen-3-yl)piperidine-2,4-dione (120 mg, 0.24 mmol) was added and the reaction mixture was stirred for 1.5 hr at 0° C. The reaction was stopped by the addition of aqueous HCl 1M (5 mL). The aqueous phase was extracted with ethyl acetate (3×5 mL) and the combined organic phases were dried over Na₂SO₄, filtered and concentrated under reduced pressure. The crude material was purified by flash column chromatography (SiO2, heptane/ethyl acetate: 4/1 to 2/1 to 1/1) to give 3-((2-chlorophenyl)thio)-6-(6-(oxetan-3-yloxy)pyridin-2-yl)-6-(thiophen-3-yl)piperidine-2,4-dione in 61% yield.

¹H NMR (400 MHz, MeOH-d4): δ=7.81-7.78 (m, 1H), 7.44 (dd, J=5.1, 3.0 Hz, 1H), 7.26-7.22 (m, 3H), 7.11 (dd, J=5.1, 1.4 Hz, 1H), 6.95 (td, J=7.5, 1.4 Hz, 1H), 6.89 (d, J=8.2 Hz, 1H), 6.75 (td, J=8.0, 1.3 Hz, 1H), 5.92 (dd, J=8.0, 1.4 Hz, 1H), 5.6 (m, 1H), 4.64 (dd, J=7.2, 5.5 Hz, 2H), 4.59 (dd, J=7.3, 5.5 Hz, 2H), 3.80 (d, J=16.5 Hz, 1H), 3.44 (d, J=16.5 Hz, 1H).

### Example 11—Preparation of 3-((2-chlorophenyl)thio)-6-(6-(3-fluorobenzyl)pyridin-2-yl)-6-(thiophen-3-yl)piperidine-2,4-dione (Example 5 in WO 2015/142903)

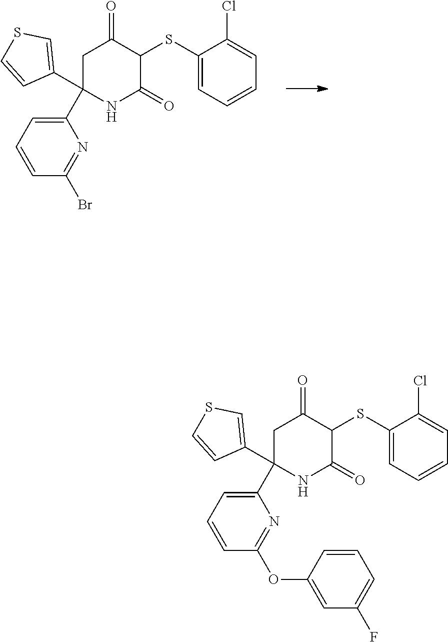 [SMILES: O=C1CC(c2ccsc2)(c2cccc(Br)n2)NC(=O)C1Sc1ccccc1Cl ; O=C1CC(c2ccsc2)(c2cccc(Oc3cccc(F)c3)n2)NC(=O)C1Sc1ccccc1Cl]

3-Fluorobenzylzinc chloride in THF (20.3 mL, 10.1 mmol) was added dropwise to a stirred mixture of 6-(6-bromopyridin-2-yl)-3-((2-chlorophenyl)thio)-6-(thiophen-3-yl)piperidine-2,4-dione (1.00 g, 2.0 mmol) and Pd(PPh₃)₄ chemdose tablet (2 μmol) in dry THF (15 mL). The reaction mixture was stirred at room temperature under an atmosphere of argon for 7 days. The reaction was then quenched by the addition of water and filtered through celite. The residue was washed with ethyl acetate and the combined filtrates were dried over sodium sulfate, filtered, and the solvent was then removed under reduced pressure. The crude mixture was then subjected to column chromatography (hexane/ethyl acetate 3/1) to provide the product (0.87 g, 83%) as an orange foamy solid. The ¹H NMR spectrum was consistent with that reported.

### Example 12—Preparation of 3-((2-chlorophenyl)thio)-6-(6-(isopentyloxy)pyridin-2-yl)-6-(thiophen-3-yl)piperidine-2,4-dione (Example 124 in WO 2015/142903)

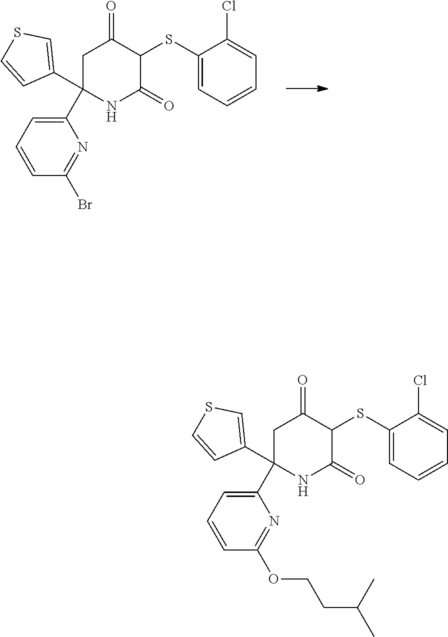 [SMILES: O=C1CC(c2ccsc2)(c2cccc(Br)n2)NC(=O)C1Sc1ccccc1Cl ; CC(C)CCOc1cccc(C2(c3ccsc3)CC(=O)C(Sc3ccccc3Cl)C(=O)N2)n1]

Sodium hydride (60% in mineral oil, 0.40 g 10.0 mmol) was washed with pentane (3×2 mL) after which THF (40 mL) was added. 3-Methyl-1-butanol (0.88 g, 10.0 mmol) was added to the resulting slurry at 0° C. and the reaction mixture was stirred at this temperature for 30 min. 6-(6-Bromopyridin-2-yl)-3-((2-chlorophenyl)thio)-6-(thiophen-3-yl)piperidine-2,4-dione (1.00 g, 2.0 mmol) was added in one portion and the mixture was then heated at reflux for 17 hr. After this time the reaction mixture was cooled to 0° C. and was subsequently quenched by adding water (20 mL). Ethyl acetate (60 mL) was then added and the mixture was neutralized to pH 7 by the addition of 1 M hydrochloric acid. The organic layer was separated and washed with brine. This was then dried over sodium sulfate, filtered, and the solvent was removed under reduced pressure. The crude mixture was then subjected to column chromatography (hexane/ethyl acetate, 2/1 to 1/1), which provided the product (0.61 g) as a pale tan solid. The ¹H NMR spectrum was consistent with that reported.

### Example 13—Coupled Diaphorase Assay

The inhibitory properties of the compounds were investigated using a coupled enzyme assay that links the lactate dehydrogenase (LDH) reaction to the production of fluorescent resorufin by diaphorase.

Human lactate dehydrogenases (LDH) catalyze the reversible interconversion between pyruvate and lactate. LDH is capable of catalyzing both the forward (pyruvate to lactate) and the reverse (lactate to pyruvate) reaction, using either NADH or NAD+ as cofactor. The reaction proceeds in either direction dependent on various factors, such as substrate availability, the presence of necessary cofactors, temperature and pH.

The coupled assay relies on the oxidation of NAD+ to NADH throughout the conversion of lactate to pyruvate by LDHC. The produced NADH serves as cofactor in the diaphorase reaction, which reduces non-fluorescent resazurin to fluorescent resorufin. Therefore, the assay indirectly monitors the rate of pyruvate production. Although the consumption or formation of NADH can be directly monitored due to the intrinsic fluorescence of the molecule (excitation: 340 nm, emission: 460 nm) there are problems linked to the direct readout method. It has been shown that many compounds in chemical libraries interfere with the assay due to fluorescent properties similar to NADH. Shifting the assay to longer wavelengths by coupling the LDH reaction to the conversion of resazurin to fluorescent resorufin by diaphorase reduces this compound interference. The assay direction was thus chosen to provide a robust and reliable assay.

For the determination of IC₅₀ values a coupled diaphorase assay was adopted from Bembenek et al. (A Fluorescence-Based Coupling Reaction for Monitoring the Activity of Recombinant Human NAD Synthetase. ASSAY and Drug Development Technologies, 2005. 3(5): 533-541). Compounds were tested in duplicates using 2-fold, 3-fold or 4-fold serial dilutions including 11 individual concentrations, starting from 5000 μM to 30 μM depending on their activity in initial tests. Positive (without compound in the presence of DMSO) and inhibition controls (oxamate, 28.7 mM final concentration in assay) or no-substrate controls representing 100% inhibition were added. Oxamate is a well characterized inhibitor of LDH that inhibits LDH enzyme activity in the mM range in vitro with high specificity (Papacostantinou et al., J. Biol. Chem. 236: 278-284, 1961). The controls allowed for the calculation of the percentage inhibition for each data point. The assay buffer consisted of 50 mM HEPES pH 7.4, 5 mM MgCl₂ and 0.05% pluronic acid F-127. Enzyme solution with final concentrations of 10 nM LDHC, as well as 0.2 U/ml diaphorase was dispensed into 384-well plates (Greiner bio-one) using a CyBi®-SELMA robotic pipettor. Compound dilutions and the enzyme were incubated for at least 15 to 20 min at room temperature. Thereafter, the substrate solution was added (final concentrations: 500 μM lactate, 150 μM NAD⁺, 3 μM resazurin). The reaction was quenched by the addition of a stop solution (final concentrations: 20 mM EDTA, 400 mM NaCl, 40 mM pyruvate). Fluorescence was read out after 5 min of incubation at an excitation wavelength of 560 nm and an emission wavelength of 590 nm on a BMG labtech FLUOstar OPTIMA or a Perkin Elmer Victor X plate reader.

A counter screen was employed to remove false positives that only inhibit the diaphorase reaction. Therefore, an enzyme solution only containing diaphorase was incubated with the compound dilution series. A substrate solution leading to final concentrations of 15 μM NADH and 3 μM resazurin was added and the assay was performed as described above. A substrate solution containing only resazurin was used as 100% inhibition control. Fluorescence data was normalized to DMSO and inhibition controls resulting in percentage inhibition for every compound concentration. Dose response curves were fitted in KaleidaGraph (www.synergy.com) or Dotmatics software package (www.dotmatics.com) using a standard 4-parameter fit (Levenberg-Marquardt fitting procedure), resulting in IC₅₀ values for the test compounds. Results are presented in Table 1.

TABLE 1

| | | IC₅₀ |
|---|---|---|
| | | LDHC |
| Example No. | Compound | [μM] |
| 1 | 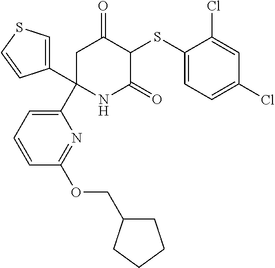 [SMILES: O=C1CC(c2ccsc2)(c2cccc(OCC3CCCC3)n2)NC(=O)C1Sc1ccc(Cl)cc1Cl] | − − |
| 2 | 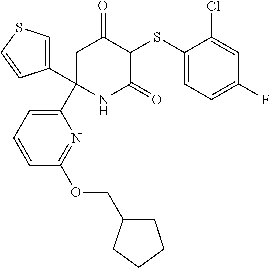 [SMILES: O=C1CC(c2ccsc2)(c2cccc(OCC3CCCC3)n2)NC(=O)C1Sc1ccc(F)cc1Cl] | + |
| 3 | 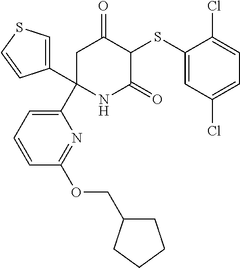 [SMILES: O=C1CC(c2ccsc2)(c2cccc(OCC3CCCC3)n2)NC(=O)C1Sc1cc(Cl)ccc1Cl] | + |
| 4 | 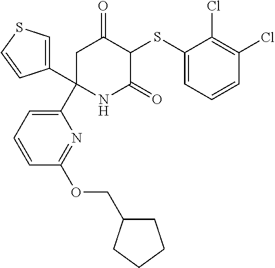 [SMILES: O=C1CC(c2ccsc2)(c2cccc(OCC3CCCC3)n2)NC(=O)C1Sc1cccc(Cl)c1Cl] | − − |
| 5 | 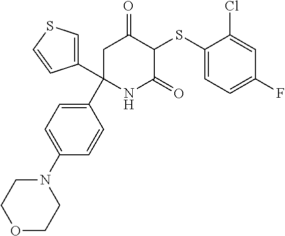 [SMILES: O=C1CC(c2ccc(N3CCOCC3)cc2)(c2ccsc2)NC(=O)C1Sc1ccc(F)cc1Cl] | + |
| 6 | 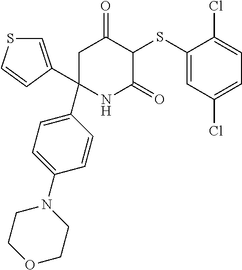 [SMILES: O=C1CC(c2ccc(N3CCOCC3)cc2)(c2ccsc2)NC(=O)C1Sc1cc(Cl)ccc1Cl] | + |
| 7 | 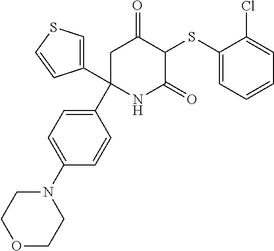 [SMILES: O=C1CC(c2ccc(N3CCOCC3)cc2)(c2ccsc2)NC(=O)C1Sc1ccccc1Cl] | + |
| 8 | 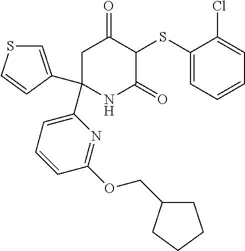 [SMILES: O=C1CC(c2ccsc2)(c2cccc(OCC3CCCC3)n2)NC(=O)C1Sc1ccccc1Cl] | + |
| 9 | 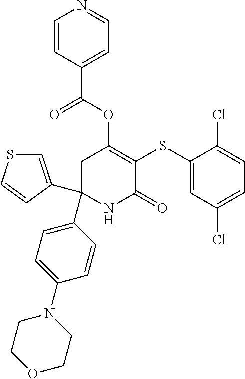 [SMILES: O=C1NC(c2ccc(N3CCOCC3)cc2)(c2ccsc2)CC(OC(=O)c2ccncc2)=C1Sc1cc(Cl)ccc1Cl] | + |
| 10 | 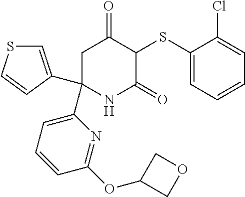 [SMILES: O=C1CC(c2ccsc2)(c2cccc(OC3COC3)n2)NC(=O)C1Sc1ccccc1Cl] | − |
| 11 | 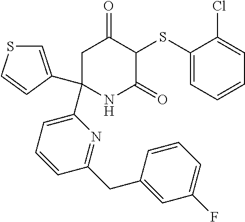 [SMILES: O=C1CC(c2ccsc2)(c2cccc(Cc3cccc(F)c3)n2)NC(=O)C1Sc1ccccc1Cl] | + |
| 12 | 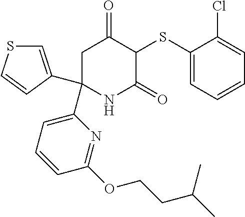 [SMILES: CC(C)CCOc1cccc(C2(c3ccsc3)CC(=O)C(Sc3ccccc3Cl)C(=O)N2)n1] | + |
| 6 Enantiomer 1 | 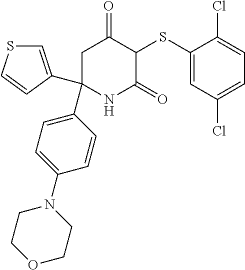 [SMILES: O=C1CC(c2ccc(N3CCOCC3)cc2)(c2ccsc2)NC(=O)C1Sc1cc(Cl)ccc1Cl] | − |
| 6 Enantiomer 2 | 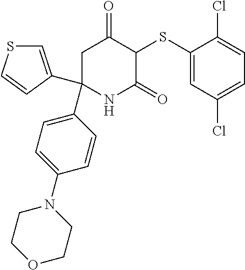 [SMILES: O=C1CC(c2ccc(N3CCOCC3)cc2)(c2ccsc2)NC(=O)C1Sc1cc(Cl)ccc1Cl] | − − |
| 7 Enantiomer 1 | 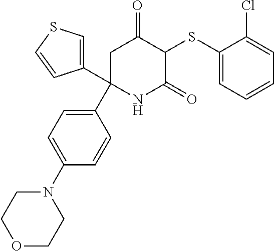 [SMILES: O=C1CC(c2ccc(N3CCOCC3)cc2)(c2ccsc2)NC(=O)C1Sc1ccccc1Cl] | + |
| 7 Enantiomer 2 | 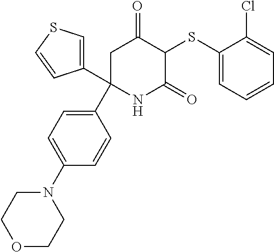 [SMILES: O=C1CC(c2ccc(N3CCOCC3)cc2)(c2ccsc2)NC(=O)C1Sc1ccccc1Cl] | − |

+ >0.5 to 10 μM
− >10 to 50 μM
− − >50 to 100 μM
− − − >100 μM

### Example 14−Sperm Motility Inhibition Assay

Human sperm cells were obtained from freshly ejaculated semen from healthy donors after 3 days of abstinence. Semen samples were liquefied for 30 minutes in a shaker at 37° C. and sperm cells were purified by a two layer percoll density centrifugation (Sigma-Aldrich). Purified sperm cells were diluted and kept in Hank's Balanced Salt Solution (Sigma-Aldrich) supplied with 0.2 mM pyruvate. Gossypol (Sigma-Aldrich), a terpenoid aldehyde compound which affects sperm motility (Wichmann, et al, J. Reprod. Fert., 69: 259-264, 1983) was used as a positive control.

In motility experiments, sperm cells were treated with several different concentrations of compounds up to 500 μM depending on their potency. Sperm cells were incubated at 37° C. in 5% CO₂ for 1 hr and then motility and progressive motility was measured using a computer-assisted sperm analyzer (CASA, HTM-IVOS system, version 12, Hamilton Thorne Research). Progressive cells were defined as average path velocity (VAP)>25 μm/s and straightness >80%.

Potency (IC₅₀) values were calculated for compounds based on concentration-dependent reduction in sperm motility. Results for the compounds of Examples 5, 6 and 7 are presented in FIG. 1.

1. A method of reducing sperm motility or of contraception in a subject, said method comprising the step of administering to said subject an effective amount of a compound of formula (I), a stereoisomer, tautomer, pharmaceutically acceptable salt or prodrug thereof: 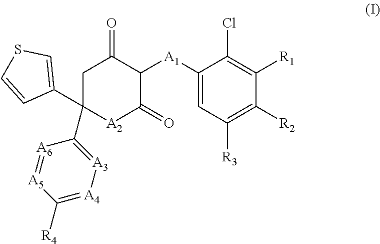
wherein:
A₁ is —O—, —CH₂—, or —S—;
A₂ is NR (wherein R is either H or C₁₋₃ alkyl);
A₃ is N or CR₅;
A₄ is N or CR₆;
A₅ is N or CR₇;
A₆ is N or CR₈;
R₁, R₂ and R₃ are independently selected from H and halogen;
R₄ is selected from:
H;
halogen;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
OR₉ in which R₉ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₁₀ in which R₁₀ is a C₃₋₈ cycloalkyl group;
R₅ is selected from:
H;
hydroxy;
C₁₋₆ alkyl; and
C₁₋₆ alkoxy;
R₆ is selected from:
H;
halogen;
C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and aryl groups;
C₁₋₆ alkoxy optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and C₃₋₈ cycloalkyl groups;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H,
—C(O)—O—C₁₋₆ alkyl, —C(O)—C₁₋₆ alkyl, amino, cyano, and nitro groups;
OR₁₁ in which R₁₁ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₂ in which R₁₂ is a C₃₋₈ cycloalkyl group;
R₇ and R₈ are independently selected from:
H;
hydroxy;
C₁₋₆ alkyl; and
C₁₋₆ alkoxy;
with the provisos that:
A₃ and A₄ are not both N at the same time; and
A₅ and A₆ are not both N at the same time.

2. A method as claimed in claim 1 comprising administering to said subject an effective amount of a compound of formula (II), a stereoisomer, or pharmaceutically acceptable salt thereof: 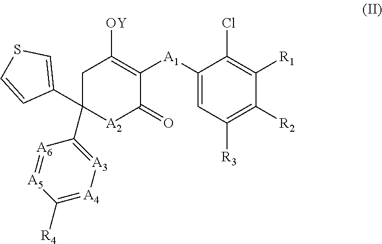
wherein A₁ to A₆ and R₁ to R₄ are as defined in claim 1; and
Y is either H or a progroup.

3. A method as claimed in claim 1 or claim 2, wherein A₁ is —S—.

4. A method as claimed in any one of claims 1 to 3, wherein A₂ is NH.

5. A method as claimed in any one of the preceding claims, wherein A₄ is CR₆.

6. A method as claimed in any one of the preceding claims, wherein A₅ is CR₇ and/or A₆ is CR₈, preferably wherein A₅ and/or A₆ is CH.

7. A method as claimed in any one of the preceding claims, wherein A₃ is N.

8. A method as claimed in any one of the preceding claims, wherein A₄ is other than CH.

9. A method as claimed in any one of the preceding claims, wherein R₄ is H.

10. A method as claimed in claim 1 comprising administering to said subject an effective amount of a compound of formula (III), a stereoisomer, pharmaceutically acceptable salt, or prodrug thereof: 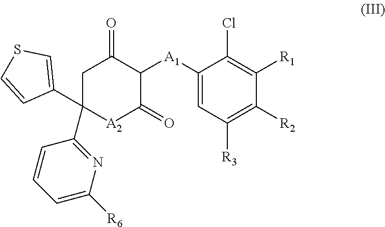
wherein A₁, A₂, R₁ to R₃ and R₆ are as defined in any one of claims 1, 3 and 4, preferably wherein R₆ is other than H.

11. A method as claimed in any one of the preceding claims, wherein
R₆ is selected from any of the following:
halogen;
C₁₋₆ alkyl optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and aryl groups;
C₁₋₆ alkoxy optionally substituted by one or more substituents selected from the group consisting of halogen, hydroxy, C₁₋₆ alkoxy, C₁₋₆ haloalkyl, C₁₋₆ hydroxyalkyl, amino, cyano, nitro, and C₃₋₈ cycloalkyl groups;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
OR₁₁ in which R₁₁ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₁₂ in which R₁₂ is a C₃₋₈ cycloalkyl group.

12. A method as claimed in any one of the preceding claims, wherein R₆ is halogen (e.g. Br or Cl, preferably Br), or an optionally substituted C₁₋₆ alkoxy group.

13. A method as claimed in any one of the preceding claims, wherein R₆ is a C₁₋₆ alkoxy group substituted by a C₃₋₈ cycloalkyl group, e.g. substituted by unsubstituted cyclopentyl.

14. A method as claimed in any one of claims 1 to 6, 8, and 11 to 13, wherein A₃ is CH and R₄ is other than H, preferably wherein R₄ is an optionally substituted 4- to 6-membered heterocyclic ring.

15. A method as claimed in claim 1 comprising administering to said subject an effective amount of a compound of formula (IV), a stereoisomer, pharmaceutically acceptable salt, or prodrug thereof: 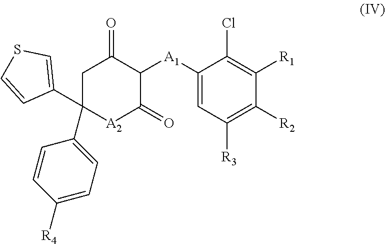
wherein A₁, A₂, R₁ to R₃ are as defined in any one of claims 1, 3 and 4; and
R₄ is selected from any of the following:
halogen;
a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups;
OR₉ in which R₉ is a 4- to 6-membered heterocyclic ring optionally substituted by one or more substituents selected from the group consisting of halogen, hydroxy, C₁₋₆ alkyl, C₁₋₆ alkoxy, C₁₋₆ haloalkyl, C₁₋₆ hydroxyalkyl, —CO₂H, —C(O)—O—C₁₋₆ alkyl, —C(O)—C₁₋₆ alkyl, amino, cyano, and nitro groups; and
OR₁₀ in which R₁₀ is a C₃₋₈ cycloalkyl group;

16. A method as claimed in any one of the preceding claims, wherein at least one of R₁, R₂ and R₃ is halogen, preferably wherein one or two of R₁, R₂ and R₃ are halogen.

17. A method as claimed in any one of the preceding claims, wherein R₁ is halogen and R₂ and R₃ are H, wherein R₂ is halogen and R₁ and R₃ are H, or wherein R₃ is halogen and R₁ and R₂ are H.

18. A method as claimed in any one of the preceding claims, wherein either R₁ or R₃ is —Cl, or R₂ is —F.

19. A method as claimed in any one of claims 1 to 15, wherein R₁, R₂ and R₃ are each H.

20. A method as claimed in any one of the preceding claims, wherein said compound is selected from the following:
6-[6-(cyclopentylmethoxy)pyridin-2-yl]-3-[(2,4-dichlorophenyl)sulfanyl]-6-(thiophen-3-yl)piperidine-2,4-dione;
3-[(2-chloro-4-fluorophenyl)sulfanyl]-6-[6-(cyclopentylmethoxy)pyridin-2-yl]-6-(thiophen-3-yl)piperidine-2,4-dione;
6-[6-(cyclopentylmethoxy)pyridin-2-yl]-3-[(2,5-dichlorophenyl)sulfanyl]-6-(thiophen-3-yl)piperidine-2,4-dione;
6-[6-(cyclopentylmethoxy)pyridin-2-yl]-3-[(2,3-dichlorophenyl)sulfanyl]-6-(thiophen-3-yl)piperidine-2,4-dione;
3-((2-chloro-4-fluorophenyl)thio)-6-(4-morpholinophenyl)-6-(thiophen-3-yl)piperidine-2,4-dione;
3-((2,5-dichlorophenyl)thio)-6-(4-morpholinophenyl)-6-(thiophen-3-yl)piperidine-2,4-dione;
3-((2-chlorophenyl)thio)-6-(4-morpholinophenyl)-6-(thiophen-3-yl)piperidine-2,4-dione;
3-((2-chlorophenyl)thio)-6-(6-(cyclopentylmethoxy)pyridin-2-yl)-6-(thiophen-3-yl)piperidine-2,4-dione;
5-((2,5-dichlorophenyl)thio)-2-(4-morpholinophenyl)-6-oxo-2-(thiophen-3-yl)-1,2,3,6-tetrahydropyridin-4-yl isonicotinate
3-((2-chlorophenyl)thio)-6-(6-(oxetan-3-yloxy)pyridin-2-yl)-6-(thiophen-3-yl)piperidine-2,4-dione
3-((2-chlorophenyl)thio)-6-(6-(3-fluorobenzyl)pyridin-2-yl)-6-(thiophen-3-yl)piperidine-2,4-dione
3-((2-chlorophenyl)thio)-6-(6-(isopentyloxy)pyridin-2-yl)-6-(thiophen-3-yl)piperidine-2,4-dione;
and their stereoisomers, tautomers, pharmaceutically acceptable salts, and prodrugs thereof.

21. A method as claimed in any one of claims 1 to 20, wherein the compound is administered together with one or more pharmaceutically acceptable carriers, excipients and/or diluents.

22. A method as claimed in any one of claims 1 to 21, wherein the compound is administered orally, parentally, topically or intradermally; preferably orally.

23. A method as claimed in any one of claims 1 to 22, wherein the subject is a mammal, preferably a human.

24. A method as claimed in any one of claims 1 to 23, wherein the subject is a male.

25. A compound as defined in any one of claims 1 to 20, a stereoisomer, tautomer, pharmaceutically acceptable salt or prodrug thereof, for use in a method of reducing sperm motility or for use as a contraceptive, preferably a male contraceptive.

26. Use of a compound as defined in any one of claims 1 to 20, a stereoisomer, a tautomer, pharmaceutically acceptable salt or prodrug thereof, in the manufacture of a medicament for use in a method of reducing sperm motility or for use as a contraceptive, preferably a male contraceptive.
